(19) United States
(12) Patent Application Publication
(10) Pub. No.: US 2005/0118148 A1
(43) Pub. Date: Jun. 2, 2005

(54) Compositions and methods related to mammalian Maf-A

(76) Inventors: Roland Stein, Nashville, TN (US); Taka-aki Matsuoka, Osaka (JP); Li Zhao, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 10/741,158

(22) Filed: Dec. 19, 2003

Related U.S. Application Data
(60) Provisional application No. 60/435,877, filed on Dec. 20, 2002.

(51) Int. Cl. A61K 48/00; C12N 5/08; C12N 15/85; C12N 15/86

(52) U.S. Cl. 424/93.21; 435/370; 435/455; 435/456

(57) ABSTRACT

Various embodiments of the invention include methods of generating a β-like cell comprising providing an expression cassette comprising a nucleic acid sequence encoding MafA under the control of a heterologous promoter and transferring the expression cassette into a non-insulin producing cell, wherein the expression of the MafA in the cell converts the cell into a β-like cell.

The present application claims priority on co-pending U.S. Provisional Patent Application Ser. No. 60/435,877 filed Dec. 20, 2002. The entire text of the above-referenced disclosure is specifically incorporated by reference herein without discretion. The government own rights in the present invention pursuant to grant number P01 DK42502 from the National Institutes of Health.

### BACKGROUND OF THE INVENTION

1. Field of the Invention

The present invention relates generally to the fields of molecular biology and therapeutics. More particularly, it concerns generating β like cells by introducing a MafA expression cassette.

2. Description of Related Art

Diabetes Mellitus, a disease of relative or absolute deficiency of insulin production, is a major metabolic disease that results in severe complications in subjects suffering from the disease. Since Insulin production in pancreatic β cells is typically reflected in the level of transcription from the insulin gene, the regulatory mechanisms of insulin transcription can be used to develop new approaches for diabetes therapy.

The insulin gene promoter contains various conserved insulin enhancer elements, for example, A3 (−201 to −196 base pair) (Karlsson, 1987, 1989; German, 1994; Peshavaria, 1994), C1/RIPE3b1 (−118 to −107 base pair) (Shieh, 1991; Sharma, 1994), and E1 (−100 to −91 base pair) (Whelan, 1990; Shieh, 1991; German, 1994) that are involved in the cell-specific regulation and glucose-responsive transcription/expression of the insulin gene. The protein or protein complexes that are known to bind the A3 and/or E1 enhancer elements include PDX-1, islet β cell-enriched homeodomain protein, and a heterodimer of ubiquitously expressed basic helix-loop-helix (bHLH) family members E2A/HEB and islet β cell-enriched bHLH protein NeuroD/BETA2 (Ohlsson, 1993; Petersen, 1998; Peshavaria, 1994; Naya, 1995). The paired domain transcription factor Pax-6 also works as an insulin gene transcription factor through a C2 element (−317 to −311 base pairs) (Sander, 1997). Strikingly, these proteins regulate gene expression within islet cell types during pancreogenesis, the formation and development of the pancreas.

Inactivating mutations in the PDX-1 locus affects a very early developmental step of pancreogenesis preventing both exocrine and endocrine pancreas formation (Jonsson, 1994; Offield, 1996). Whereas, null mutations in BETA2 (Naya, 1997) and Pax-6 (St-Onge, 1997) proteins affect later, but distinct stages of endocrine islet cell formation. Moreover, the ability of islet β cells to produce insulin is compromised in type 2 diabetes mellitus patients with an inactivating mutation within one allele of either the PDX-1 (Hani, 1999; Stoffers, 1997b), BETA2 (Malecki, 1999) or Pax-6 (Yasuda, 2002) genes. Collectively, these results established a central role for each of the isolated insulin gene transcription factors in islet cell development and function.

The C1/RIPE3b1 binding protein, RIPE3b1 activator, has not been fully investigated, compared with PDX-1, BETA2, and Pax-6, it was already reported that RIPE3b1 activator is a pancreatic β cell enriched DNA-binding protein(s), whose levels are regulated by glucose in parallel with insulin gene transcription (Shieh, 1991; Sharma, 1994; Zhao, 2000). The inventors have recently reported that RIPE3b1 binding activity was decreased when RIPE3b1 activator was incubated with endogenous β cell phosphatase, calf intestinal alkaline phosphatase (CIAP) or a brain-enriched phosphatase preparation (BPP), which were prevented by tyrosine phosphatase inhibitors (Zhao, 2000; Matsuoka, 2001). In addition, the inventors have demonstrated RIPE3b1 activator is a phosphorylatable protein (Matsuoka, 2001). As previous reports show, DNA binding activity of PDX-1 is regulated by its phosphorylation status (Macfarlane, 1994; Macfarlane, 1997), and NeuroD and its family NeuroD2 are also phosphorylated proteins (personal communication; Kume, 1998), which is consistent with glucose stimulating various phosphorylation cascades in pancreatic β cells and promoting insulin production (Leibiger, 1998; Macfarlane, 1997; Benes, 1999). The general characteristics of RIPE3b1 activator suggests that it has a role in not only pancreatic β cell function, including insulin transcription, but also in pancreogenesis. The further characterization of the RIPE3b1 activator and its method of action will be a significant step in the study of the etiology of diabetes and molecular pathways of pancreogenesis. There remains a need for identifying the molecular components of RIPE3b1 and utilizing these components in methods for treating diabetes.

### SUMMARY OF THE INVENTION

Various embodiments of the invention include methods of generating a β-like cell comprising providing an expression cassette comprising a nucleic acid sequence encoding MafA under the control of a heterologous promoter and transferring the expression cassette into a non-insulin producing cell, wherein the expression of the MafA in the cell converts the cell into a β-like cell. The cell may be a progenitor cell and in particular the cell may be a pancreatic progenitor cell. The cell may immortalized. An immortalized cell may or may not be susceptible to a cell kill agent. The immortalized cell may further comprise a heterologous nucleic acid segment encoding a polypeptide that renders the cell susceptible to the cell kill agent. The cell kill agent may or may not be under the control of an inducible promoter.

In certain embodiments, the expression cassette is comprised in a viral or a non-viral vector. In some embodiments the non-viral vector is as a plasmid. In other embodiments the expression cassette is comprised in a viral vector. The viral vector may be an adenovirus, a retrovirus, a herpes-simplex virus, a vaccinia virus or an adeno-associated virus. In various embodiments, the expression cassette is transferred into the cell by a non-viral delivery system. The non-viral delivery system may be calcium phosphate precipitation, DEAE-dextran, electroporation, direct microinjection, DNA-loaded liposomes, cell sonification, gene bombardment using high velocity microprojectiles or receptor-mediated transfection. In some embodiments, a promoter is further defined as an inducible promoter or a metallothionine promoter. It is specifically contemplated that a promoter be an inducible promoter. An inducer of an inducible promoter may be provided to the cell.

In various embodiments, a method of generating a β-like cell comprises providing a MafA protein comprising a nuclear localization signal; and contacting a cell with a sufficient amount of the MafA protein; wherein contacting the cell with a sufficient amount of the MafA protein converts the cell into a β-like cell.

In other embodiments, an implantable device for treating diabetes in a subject is contemplated. The implantable device may comprise a receptacle suitable for holding live cells; a cell-impermeable membrane operably fixed to the receptacle so as to confine the cells within the receptacle, wherein the membrane is permeable to insulin, regulatory signals that regulate the production of insulin and other factors necessary for the survival of the cells. In certain embodiments the device is suitable for placement in a human body. In particular embodiments a regulatory signal is glucose and the factors may comprise nutrients.

In some embodiments, a method of providing regulated insulin production to a subject comprising providing an effective amount of a composition comprising a β-like cell to a subject, wherein the β-like cell comprises an expression cassette comprising a nucleic acid sequence encoding MafA under the control of a heterologous promoter is contemplated. The heterologous promoter may be an inducible promoter. The expression cassette may be maintained in an episomal form or integrated into the cell genome of a host cell. The subject may a human.

In other embodiments, a method of treating diabetes in a subject comprising providing an effective amount of a composition comprising a β-like cell to a subject, wherein the β-like cell comprises an expression cassette comprising a nucleic acid sequence encoding MafA under the control of a heterologous promoter is also specifically contemplated. In certain embodiments the subject is a human. Diabetes treated may be Type I or Type II diabetes. A heterologous promoter may or may not be an inducible promoter. In certain embodiments the expression cassette is maintained in an episomal form or integrated into the cell genome.

In some embodiments, a composition comprising a β-like cell, wherein the β-like cell comprises an expression cassette comprising a nucleic acid sequence encoding MafA under the control of a heterologous promoter is contemplated.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H. RIPE3b1 activator has two isoelectric points. FIGS. 1A-1B—Analysis of βTC-3 nuclear proteins separated by 2D gel electrophoresis. FIGS. 1C-1D—The RIPE3b1 DNA binding activity detected in the eluted protein(s) from spots on 2D gel indicated by South-Western blotting. FIG. 1E—Competition assay on gel-shift analysis showed eluted protein bind C1/RIPE3b1 probe specifically. FIG. 1F—Purification of RIPE3b1 binding protein. FIGS. 1G-1H—South-Western blotting analysis indicated 2 protein spots at pH 7.0 and 4.5 on 46 kDa were observed on Coomassie stained 2D gel.

FIGS. 2A-2B. FIG. 2A—Identification and Cloning of RIPE3b1 activator. FIG. 2B—Large Mafs can bind C1/RIPE3b1 cis-element specifically.

FIG. 3. Binding of L-Maf, MafB and c-Maf to C1/RIPE3b1 probe were competed by wild type competitor but not by −108/111 mutant.

FIG. 4. Maf western blotting with anti-Maf antibodies.

FIGS. 5A-5B. cMaf recognize RIPE3b1 complex. FIG. 5A—αMaf entirely super-shifted RIPE3b1 complex in βTC-3 nuclear extract as well as L-Maf and c-Maf complex, and weakly super-shifted MafB complex. FIG. 5B—Antibody effects on RIPE3b1 complex in βTC3 nuclear extract were the same as that in islet nuclear extract.

FIG. 6. Maf s effects on insulin promoter. L-Maf, MafB and c-Maf significantly activated insulin promoter, on the other hand, the mutation in −108/−111 decreased insulin promoter activity brought by these Mafs. Neither S14A, S65A, nor double mutant S14A/S65A indicated significant difference from wild L-Maf on insulin promoter activity.

FIG. 7. RT-PCR analysis performed with each large Maf specific primer sets. Except NRL, L-Maf, MafB and c-Maf were reproducibly amplified from mouse islet, βTC-3 and αTC6 cell RNAs by RT-PCR.

FIG. 8. Maf binds insulin promoter/enhancer region in vivo.

FIGS. 9A-9B. Maf protein's expression in mouse islet. FIG. 9A—Double staining with αMaf and insulin indicate large Maf protein abundance in nuclei of islet β cells but not exocrine cells. FIG. 9B—Double staining with αMaf and glucagon showe at least L-Maf and c-Maf, which are recognized well by αMaf, are not abundant in glucagon producing cell.

FIGS. 10A-10B. The conserved B4 and B5 sequence blocks regulate PstBst-mediated activation in β cells. FIG. 10A—A schematic diagram illustrating the position of the −2560/−1880 bp PstBst region in the mouse pdx-1 gene. The location of the Area I and Area II control regions and the characterized conserved block mutants within Area II are also shown. The Foxa2 and Pax6 control elements were characterized previously. FIG. 10B—The normalized activity of the transfected mutant Area II:pTKCAT and PstBst:pTKCAT constructs is expressed as a percentage activity of the wild type Area II and PstBst reporter±Standard error of the mean (SEM).

FIGS. 11A-11C. B4 and B5 comprise a single element that binds a β cell-enriched factor. FIG. 11A—The B4, B5 and B4/5 probe sequences are shown. The conserved B4 and B5 block sequences are in bold. FIG. 11B—Gel shift binding reactions were conducted with the B4, B5, or B4/5 probe using βTC-3 and Min6 nuclear extracts in the presence of a fold molar excess of unlabeled wild type (wt) competitor to probe. The complexes labeled A and B are discussed in the text. FIG. 11C—Nuclear extracts from β (βTC-3, Ins-1, Min6, HIT-T15) and non-β (αTC-6, RC2-E10, NCB20, MDCK, BHK, NIH-3T3, and H4IIE) cell lines as well as rat liver (rliver) were analyzed for B4/5 binding activity.

FIG. 12. The RIPE3b1 complex contains a protein(s) of approximately 46 kDa. Min6 nuclear extracts were electrotransferred from an SDS-PAGE onto an Immobilon PVDF membrane. The proteins were eluted from membrane slices and assayed for B4/5 and InsC1 binding activity. Binding specificity was determined by competition with a 10-fold excess unlabeled B4/5 or InsC1. Each fraction represents a different molecular weight range. The position of the complex A detected in unfractionated Min6 nuclear extracts is indicated.

FIGS. 13A-13B. B4/5 and InsC1 form similar β cell protein-DNA complexes. FIG. 13A—The sequences of B4/5 and InsC1 probes. B4 and B5 sequences are contained within the blocked region, and mutated sequences in InsC1mt1 and InsC1mt3 are shown. FIG. 13B—Binding reactions were conducted with Min6 and βTC-3 nuclear extract. The molar ratio of the wildtype or mutant competitor to the labeled probe is shown.

FIGS. 14A-14B. Complex A binding to B4/5 appears is sensitive to tyrosine dephosphorylation. FIG. 14A—B4/5 and Ins C1 binding assay with Min6 nuclear extracts were incubated at either 4° C. or at 30° C. either alone or in the presence of CIAP, CIAP+10 mM Na₃VO₄, or CIAP+10 mM NaPPi. FIG. 14B—βTC-3 nuclear extract was immunoprecipitated with either the anti-phosphotyrosine antibody, 4G10, or normal mouse IgG. The immunoprecipitated protein and whole nuclear extract were then fractionated by SDS-PAGE and transferred to PVDF membranes. Protein fraction 1 (53.7-62.7 kDa), 2 (41.7-53.6 kDa), and 3 (29.9-41.6 kDa) were eluted and used in B4/5 and Ins C1 gel shift assays along with unfractionated βTC-3 nuclear extract.

FIGS. 15A-15B. InsC1 can substitute for B4/5 in driving Area II activation.

FIG. 15A—The sequence of human (h) and mouse (m)B4/5 is compared to mInsC1. The nonconserved bases in the human and mouse B4/5 are in lowercase letters. The bases shown to be important for A/RIPE3b1 factor binding in methylation interference assays are indicated with asterisks (InsC1); B4/5, data not shown). FIG. 15B—mInsC1 (−124/−105) was inserted into PstBst (PB):pTKCAT in place B4/B5 (−2100/−2082) and the activity compared to other PB:pTKCAT constructs in transfected Min6 and NIH-3T3 cells. The normalized activity of the mutant PB:pTKCAT is expressed as the percentage of the wild type ±SEM.

FIG. 16. RIPE3b1/Maf binds to the Area II region in vivo. Cross-linked chromatin from βTC-3 cells was incubated with a polyclonal antibody raised to N-terminal sequences in c-Maf that are conserved in other L-Mafs, including MafA, MafB, and Nr1. The immunoprecipitated DNA was analyzed by PCR for Area II and PEPCK transcriptional regulatory sequences. As controls, PCR reactions were run on total input chromatin, with no DNA, and with DNA obtained after precipitating with rabbit IgG.

### DESCRIPTION OF ILLUSTRATIVE EMBODIMENTS

### I. Present Invention

Various compositions and methods of the invention include a Maf polypeptide (i.e., a RIPE3b1 binding protein) or a nucleic acid encoding a Maf polypeptide. In certain embodiments of the invention, the RIPE3b1 binding protein is a member of the large Maf family. In particular embodiments, the Maf protein is a MafA protein. Polypeptides of the invention are expressed in pancreatic islet cells and can activate the insulin promoter through C1/RIPE3b1 cis-element. The presence of a Maf polypeptide contributes not only to insulin transcription, but also development of pancreas.

As described above, pancreatic β cell-specific and glucose-regulated transcription of the insulin gene is principally mediated through the transcription factors that act upon the A3 (−201 to 196 base pair), C1/RIPE3b1 (−118 to −107 base pair), and E1 (−100 to −91 base pair) elements of the insulin gene. As a step toward further understanding of function of RIPE3b1, the inventors have purified a RIPE3b1 binding protein from βTC-3 cell nuclear extract by using modified DNA affinity purification, oligonucleotide trapping method. The purified RIPE3b1 binding fractions were separated further by two-dimentional (2D) electrophoresis, which gave two protein spots, pH 7.0 and 4.5 on 46 kDa, possessing RIPE3b1 binding activity. Analysis of these 2 spots by mass spectrometry detected 2 amino- and 6 carboxy-terminal peptides which match a mouse homolog of the chicken L-Maf/quail MafA protein. The inventors have shown that large Mafs, L-Maf, MafB and c-Maf selectively bound the C1/RIPE3b1 element, and RIPE3b1 complex was super-shifted by anti-large Maf antibody (αMaf) on gel-shift assay, in addition, these large Mafs activate insulin gene promoter through C1/RIPE3b1 element on reporter gene analysis. The inventors also showed that αMaf detected islet β-cells enriched protein on immunohistochemistry, in fact, L-Maf appeared to be representative from results of other Maf antibodies and RT-PCR. Collectively, these findings demonstrated large Maf is a RIPE3b1 activator. In certain embodiments, a Maf is used to activate the RIPE3b1 element.

### II. Maf Protein Family

A variety of developmental roles and transcriptional targets has been proposed for Maf transcription factors. The v-maf oncogene is the earliest described member of the family (Nishizawa et al., 1989). Large Maf subfamily members (c-Maf (cellular Maf) (Kataoka, 1993), L-Maf/MafA (Ogino, 1998; Benkhelifa, 1998), MafB (Kataoka, 1994; Cordes, 1994), and Nrl (neural retina leucine zipper) (Swaroop, 1992)) contain an activation domain at the N terminus, whereas small Maf subfamily members (MafF, MafK, and MafG) lack a distinct activation domain (Blanks and Andrews, 1997). Maf transcription factors share structural similarity both within and outside the basic leucine zipper domain and bind common recognition elements, 12-O-tetradecanoylphorbol 13-acetate type Maf response element or cyclic AMP response element type (Kataoka et al., 1994; Kerppola et al., 1994). Homo- and heterodimerization through leucine zipper domains is one of the most important mechanisms underlying transcriptional regulation by bZip factors. All the Maf family members can form heterodimers with other bZip factors like Fos and Jun, and these heterodimers are different in their DNA binding specificity from Maf homodimers or AP-1 complexes (Kataoka et al., 1994, Kerppola et al., 1994). BZip transcription factors are also able to interact with unrelated transcription factors like glucocorticoid receptors or Ets family members (Jonat et al., 1990, Basuyaux et al., 1997). In the case of c-Maf, interaction with the transcription factor c-Myb plays a role during myeloid cell differentiation (Hedge et al., 1998), whereas MafB interaction with c-Ets-1 represses its transcriptional activity, resulting in the inhibition of erythroid cells differentiation (Sieweke et al., 1996). Recently, Maf family members were shown to associate with a set of Hox proteins, resulting in the inhibition of Maf DNA binding, transactivation, and transforming activities (Kataoka et al., 2001). As a MARE has TRE or CRE, it was expected that Maf is regulated in a phosphorylation dependent manner. Recently, several reports indicated large Mafs are regulated under the kinase cascade and are phosphoproteins has been reported (Benkhelifa, 2001; Civil, 2002; Swain, 2001).

Maf proteins have been implicated in the control of development and differentiation, such as optic development (Kim, 1999; Kawauchi, 1999; Ring, 2000; Ogino, 1998; Reza, 2002; Cordes, 1994; Ishibashi, 2001; Bessant, 1999) nervous system (Cordes, 1994) and blood cell differentiation (Kelly, 2000; Sieweke, 1996) through the binding and activation of MARE in promoter region of target genes. The large Mafs developmental function have been extensively studied in the eye. As examined by in situ hybridization using mouse embryo, c-Maf expression is detectable in the lens vesicle between E10.5 and E11, while MafB is found in lens epithelial cells in E10.5 to E 14.5 embryos but not in the lens fiber cells, and NRL is not expressed during the early stage of the lens development (Ring, 2000; Kawauchi, 1999; Liu, 1996). Expression of mammalian L-Maf has not been demonstrated in the eye, but, since L-Maf is expressed in the epithelial placode which is the initiation of lens differentiation in chicken embryo (Ogino, 1998), it is plausible that L-Maf is expressed earlier than other large Mafs. These different expression patterns during eye development suggest that every large Maf has unique functions and targets, although all large Mafs contribute to eye formation and binds similar DNA consensus sequence, MARE. In fact, knockout study or mutation of each large Maf showed different phenotype in the eye (Kim, 1999; Kawauchi, 1999; Ring, 2000; Ogino, 1998; Reza, 2002; Cordes, 1994; Bessant, 1999). Although expression of L-Maf, MafB and c-Maf was found in pancreatic islet by RT-PCR, each Maf likely has a different role in the islet as well as in the eye.

### III. Nucleic Acid Compositions

Also contemplated by the present invention are nucleic acids encoding MafA polypeptides and fragments thereof. The nucleic acid sequence for human MafA is provided as SEQ ID NO:1 and mouse MafA is provided as SEQ ID NO:3.

Certain embodiments of the present invention involve the synthesis and/or mutation of at least one isolated nucleic acid molecule, such as recombinant expression vectors encoding all or part of the amino acid sequences, such as those shown in SEQ ID NO: 2 and 4. Embodiments of the invention also involve the creation and use of recombinant host cells through the application of DNA recombinant technology, that express one or more MafA peptides or polypeptides. In certain aspects, a nucleic acid encoding a MafA peptides or polypeptides or a modulator of insulin gene transcription or β cell like differentiation comprises a wild-type or a mutant nucleic acid. The nucleic acid compositions can, for example, be used in an assay for modulators of insulin transcription or for modulators of β cell activity.

Because of the degeneracy of the genetic code, many other nucleic acids also may encode a given MafA. For example, four different three-base codons encode the amino acids alanine, glycine, proline, threonine and valine, while six different codons encode arginine, leucine and serine. Only methionine and tryptophan are encoded by a single codon. A table of amino acids and the corresponding codons is presented herein for use in such embodiments (Table 1).

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In order to generate any nucleic acid encoding MafA, one need only refer to the preceding codon table. Substitution of the natural codon with any codon encoding the same amino acid will result in a distinct nucleic acid that encodes MafA or a variant thereof. As a practical matter, this can be accomplished by site-directed mutagenesis of an existing MafA gene or de novo chemical synthesis of one or more nucleic acids.

The preceding observations regarding codon selection, site-directed mutagenesis and chemical synthesis apply with equal force to the discussion of substitutional mutants in the section of peptides. Normally, substitutional mutants are generated by site-directed changes in the nucleic acid designed to alter one or more codons of the coding sequence.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to those of a purine or pyrimidine substituted by one or more of an alkyl, carboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table non-limiting, purine and pyrimidine derivatives and analogs is also provided herein below.

TABLE 2
Purine and Pyrmidine Derivatives or Analogs

| Abbr. | Modified base description |
|---|---|
| ac4c | 4-acetylcytidine |
| Chm5u | 5-(carboxyhydroxylmethyl) uridine |
| Cm | 2′-O-methylcytidine |
| Cmnm5s2u | 5-carboxymethylamino-methyl-2- |
| | thioridine |
| Cmnm5u | 5- |
| | carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2- |
| | thiouridine |
| Man q | Beta,D-mannosylqueosine |
| Mcm5s2u | 5-methoxycarbonylmethyl-2- |
| | thiouridine |
| Mcm5u | 5-methoxycarbonylmethyluridine |
| Mo5u | 5-methoxyuridine |
| Ms2i6a | 2-methylthio-N6- |
| | isopentenyladenosine |
| Ms2t6a | N-((9-beta-D-ribofuranosyl-2- |
| | methylthiopurine-6- |
| | yl)carbamoyl)threonine |
| Mt6a | N-((9-beta-D-ribofuranosylpurine-6- |
| | yl)N-methyl-carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, |
| | (acp3)u |

A nucleobase may be comprised in a nucleoside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moiety which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moiety replacing phosphodiester backbone moiety used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136 which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. 5,708,154 which describes RNA linked to a DNA to form a DNA-RNA hybrid.

In a non-limiting example, one or more nucleic acid analogs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges). Such analogs may be implemented with respect to SEQ ID NOS:1 or 3 as provided herein or variants thereof.

E. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and PCT Patent Application WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5891,625. Other modifications and uses of nucleic acid analogs are known in the art, and are encompassed by the nucleic acid encoding for apoptosis modulators. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. patent application No. 117,363 describes several alkylamino moieties used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

F. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989 and 2001, incorporated herein by reference).

G. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

H. Nucleic Acid Segments

In certain embodiments, the nucleic acid is a nucleic acid segment. As used herein, the term “nucleic acid segment,” are fragments of a nucleic acid, such as for non-limiting example, those that encode only part of a MafA peptide or polypeptide sequence. Thus, a “nucleic acid segment” may comprise any part of a gene sequence, of from about 2 nucleotides to the full length of the MafA peptide- or polypeptide-encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In a non-limiting example, nucleic acid segments may contain up to 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 nucleotides. Contiguous nucleic acids segments of SEQ ID NO: 1 or 3 may be used in the present invention. Nucleic acid segments may also contain up to 10,000, 20,000, 30,000, 50,000, 100,000, 250,000, 500,000, 750,000, to 1,000,000 nucleotides in length, as well as constructs of greater size, up to and including chromosomal sizes are contemplated for use in the present invention. Furthermore, nucleic acids, including expression constructs, may comprise at least 5, 10, 15, 20, 25, 30, 35, 40, 45, 50, 55, 60, 65, 70, 75, 80, 85, 90, 95, 100, 150, 200, 250, 300,.350, 400, 450, 500, 550, 600, 650, 700, 750, 800, 850, 900, 950, 1000, 2000, 3000, 4000, or 5000 contiguous nucleic acid residues or nucleotides from SEQ ID NO: 1 or 3.

I. Nucleic Acid Complements

The present invention also encompasses a nucleic acid that is complementary to the nucleic acid encoding for MafA polypeptide. In particular embodiments the invention encompasses a nucleic acid or a nucleic acid segment complementary to the sequence set forth in SEQ ID NO: 1 or 3. A nucleic acid is a “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

J. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high. stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present invention also concerns the isolation or creation of a recombinant construct or a recombinant host cell through the application of recombinant nucleic acid technology known to those of skill in the art or as described herein. A recombinant construct or host cell may express an MafA or insulin gene modulator protein, peptide or peptide, or at least one biologically functional equivalent thereof. The recombinant host cell may be a prokaryotic cell. In a more preferred embodiment, the recombinant host cell is a eukaryotic cell. As used herein, the term “engineered” or “recombinant” cell is intended to refer to a cell into which a recombinant gene, such as a gene encoding an MafA or insulin gene modulator, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene (i.e., they will not contain introns), a copy of a genomic gene, or will include genes positioned adjacent to a promoter not naturally associated with the particular introduced gene.

In certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like. The term “cDNA” refers to that portion of a gene that is transcribed.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, or about 20; about 21, about 22, about 23, or more; about 31, about 32, or more; about 51, about 52, about 53, or more; about 101, about 102, about 103, or more; about 151, about 152, about 153, or more; about 1,001, about 1002, or more; about 50,001, about 50,002, or more; about 750,001, about 750,002, or more; about 1,000,001, about 1,000,002, or more. Non-limiting examples of intermediate ranges include about 3 to about 32, about 150 to about 500,001, about 3,032 to about 7,145, about 5,000 to about 15,000, about 20,007 to about 1,000,003, etc. Such constructs may be implemented and used with respect to SEQ ID NO: 1 or 3.

The term “functionally equivalent codon” is used herein to refer to codons that encode the same amino acid, such as the six codons for arginine and serine, and also refers to codons that encode biologically equivalent amino acids. For optimization of expression of genes in human cells, the codons are shown in Table 1 above in preference of use from left to right. Thus, the most preferred codon for alanine is thus “GCC”, and the least is “GCG” (see Table 1, above). Codon usage for various organisms and organelles can be found at the website kazusa on the internet, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N— or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

The nucleic acids of the present invention encompass biologically functional equivalent MafA or insulin gene modulator proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine MafA or insulin gene modulator protein, polypeptide or peptide activity at the molecular level.

Fusion proteins, polypeptides or peptides may be prepared, e.g., where the coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding peptides or fusion peptides, such as, for example, peptides of from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100 amino acids in length, including such numbers of contiguous amino acids from SEQ ID NOS: 2 or 4.

As used herein an “organism” may be a prokaryote, eukaryote, virus and the like. As used herein the term “sequence” encompasses both the terms “nucleic acid” and “proteinaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

L. Vectors and Expression Constructs

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include piasmids, cosmids, viruses (bacteriophage, animal viruses, and plant viruses), and artificial chromosomes (e.g., YACs). One of skill in the art would be well equipped to construct a vector through standard recombinant techniques. (see, for example, Sambrook et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operable linked coding sequence in a particular host cell. The combination of control sequences and the nucleic acid to be expressed in a manner that expression may be achieved by introduction to a cell is termed an “expression cassette.” In addition to control sequences that govern transcription and translation, vectors and expression vectors may contain nucleic acid sequences that serve other functions as well and are described infra.

In order to express an MafA peptide or polypeptide it is necessary to provide an MafA coding region or gene in an expression vehicle. The appropriate nucleic acid can be inserted into an expression vector by standard subcloning techniques. For example, an E. coli or baculovirus expression vector is used to produce recombinant polypeptide in vitro. The manipulation of these vectors is well known in the art. In one embodiment, the protein is expressed as a fusion protein with β-gal, allowing rapid affinity purification of the protein. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these fusion systems produce recombinant protein bearing only a small number of additional amino acids, which are unlikely to affect the functional capacity of the recombinant protein. For example, both the FLAG system and the 6×His system add only short sequences, both of which are known to be poorly antigenic and which do not adversely affect folding of the protein to its native conformation. Other fusion systems produce proteins where it is desirable to excise the fusion partner from the desired protein. In another embodiment, the fusion partner is linked to the recombinant protein by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

Recombinant bacterial cells, for example E. coli, are grown in any of a number of suitable media, for example LB, and the expression of the recombinant polypeptide induced by adding IPTG to the media or switching incubation to a higher temperature. After culturing the bacteria for a further period of between 2 and 24 hours, the cells are collected by centrifugation and washed to remove residual media. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars such as sucrose into the buffer and centrifugation at a selective speed.

If the recombinant protein is expressed in the inclusion bodies, as is the case in many instances, these can be washed in any of several solutions to remove some of the contaminating host proteins, then solubilized in solutions containing high concentrations of urea (e.g. 8M) or chaotropic agents such as guanidine hydrochloride in the presence of reducing agents such as β-mercaptoethanol or DTT (dithiothreitol).

Under some circumstances, it may be advantageous to incubate the polypeptide for several hours under conditions suitable for the protein to undergo a refolding process into a conformation which more closely resembles that of the native protein. Such conditions generally include low protein concentrations less than 500 μg/ml, low levels of reducing agent, concentrations of urea less than 2 M and often the presence of reagents such as a mixture of reduced and oxidized glutathione which facilitate the interchange of disulphide bonds within the protein molecule.

The refolding process can be monitored, for example, by SDS-PAGE or with antibodies which are specific for the native molecule (which can be obtained from animals vaccinated with the native molecule isolated from parasites). Following refolding, the protein can then be purified further and separated from the refolding mixture by chromatography on any of several supports including ion exchange resins, gel permeation resins or on a variety of affinity columns.

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.). The vector carrying the gene of interest is transfected into Spodoptera frugiperda (Sf9) cells by standard protocols, and the cells are cultured and processed to produce the recombinant protein. Mammalian cells exposed to baculoviruses become infected and may express the foreign gene only. This way one can transduce all cells and express the gene in dose dependent manner.

There also are a variety of eukaryotic vectors that provide a suitable vehicle in which recombinant polypeptide can be produced. HSV has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid, for example, to generate antisense constructs.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Preferred promoters include those derived from HSV, including the U_{S}3, or the α4 promoter. Another preferred embodiment is the tetracycline controlled promoter. In particular embodiments the promoter is a metallothionine promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 3 and 4 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 3
PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α_{1-Antitrypsin}
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 4

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |

Use of the baculovirus system will involve high level expression from the powerful polyhedron promoter.

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for tile transfer of foreign genes into mammalian cells (Ridgeway, 1988; Nicholas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or a particular cellular phentotype such as β cell phenotype or (ii) to transform cells in vitro or in vivo to provide therapeutic polypeptides in a gene therapy scenario.

### 1. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

### a. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

### b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the candidate substances of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

### c. Retroviral Vectors

Retroviruses have promise as an antigen delivery vectors in vaccines of the candidate substances due to their ability to integrate their genes into the host genome, transferring a large amount of foreign genetic material, infecting a broad spectrum of species and cell types and of being packaged in special cell-lines (Miller, 1992).

In order to construct a vaccine retroviral vector, a nucleic acid (e.g., one encoding an MafA) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

### d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

### e. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### 2. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der ED, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

### 3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in at least one organism (e.g., a Human). In certain embodiments, the organism may be, but is not limited to, a prokaryote (e.g., a eubacteria, an archaea) or an eukaryote, as would be understood by one of ordinary skill in the art (see, for example, the Internet site for Arizona University on phylogeny). The host cell may be incorporated in an apparatus that allows free exchange of small molecules such as glucose and insulin. A variety of materials for implantation into an organism are known in the art.

Numerous cell lines and cultures are available for use as a host cell, and they can be obtained through the American Type Culture Collection (ATCC), which is an organization that serves as an archive for living cultures and genetic materials (on the ATCC website). An appropriate host can be determined by one of skill in the art based on the vector backbone and the desired result, i.e., transformation into a β cell like cell or cellular phenotype. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12, as well as numerous progenitor or stem cells lines or primary cultures. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using all or part of SEQ ID NO: 1 or 3.

### 4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the fill-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (on the National Center for Biotechnology information website on the Internet). The coding regions for these known genes may be amplified and/or expressed using the techniques disclosed herein or by any technique that would be know to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

### IV. MafA Protein

The protein sequence for human MafA is provided in SEQ ID NO:2 and mouse is provide in SEQ ID NO:4. In addition to the entire MafA molecule, the present invention also relates to fragments of the polypeptides that may or may not retain various of the functions described below. Fragments, including the N-terminus of the molecule, may be generated by genetic engineering of translation stop sites within the coding region (discussed below). Alternatively, treatment of the MafA with proteolytic enzymes, known as proteases, can produces a variety of N-terminal, C-terminal and internal fragments. Peptides range from 6, 7, 8, 9, 10, 15, 20, 25, 30, 35, 40, 45, and 50 residues, such as those made synthetically, up to 100, 150, 200, 250, 300, 350, 400, 450, 500 and more residues, which are conveniently produced by recombinant means or by proteolytic digestion of full length MafA. Examples of fragments may include contiguous residues of SEQ ID NO:2 of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 200, 300, 400 or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Variants of MafA

Amino acid sequence variants of the MAFA polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittie, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of MafA, but with altered and even improved characteristics.

B. Domain Switching

An interesting series of mutants can be created by substituting homologous regions of various proteins. This is known, in certain contexts, as “domain switching.”

Domain switching involves the generation of chimeric molecules using different but, in this case, related polypeptides. By comparing various MafA proteins, one can make predictions as to the functionally significant regions of these molecules. It is possible, then, to switch related domains of these molecules in an effort to determine the criticality of these regions to MafA function. These molecules may have additional value in that these “chimeras” can be distinguished from natural molecules, while possibly providing the same function.

C. Fusion Proteins

A specialized kind of insertional variant is the fusion protein. This molecule generally has all or a substantial portion of the native molecule, linked at the N— or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

D. Purification of Proteins

It will be desirable to purify MafA or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%,. about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Synthetic Peptides

The present invention also includes smaller MafA-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

F. Antigen Compositions

The present invention also provides for the use of MafA proteins or peptides as antigens for the immunization of animals relating to the production of antibodies. It is envisioned that MafA or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

G. Antibody Production

In certain embodiments, the present invention provides antibodies that bind with high specificity to the MafA polypeptides provided herein. Thus, antibodies that bind to the polypeptide of SEQ ID NO:2 are provided. In addition to antibodies generated against the full length proteins, antibodies may also be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

The methods for generating monoclonal antibodies (MAbs) generally begin along the same lines as those for preparing polyclonal antibodies. Briefly, a polyclonal antibody is prepared by immunizing an animal with an immunogenic MAFAcomposition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, Pa.); low-dose Cyclophosphamide (CYP; 300 mg/m²) (Johnson/Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified MafA protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine—resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods, which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

H. Antibody Conjugates

The present invention further provides antibodies against MafA, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins” (described in U.S. Pat. Nos. 5,686,072, 5,578,706, 4,792,447, 5,045,451, 4,664,911 and 5,767,072, each incorporated herein by reference).

Antibody conjugates are thus preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Again, antibody-directed imaging is less preferred for use with this invention.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled monoclonal antibodies of the present invention may be produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium-^{99m} by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) and ethylene diaminetetracetic acid (EDTA). Also contemplated for use are fluorescent labels, including rhodamine, fluorescein isothiocyanate and renographin.

The much preferred antibody conjugates of the present invention are those intended primarily for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

### V. Methods of Treatment

In various embodiments of the invention a variety of treatments are contemplated. Treatment may include the introduction of a MafA expression cassette into an animal, introduction of a MafA expression cassette into a cell and then administering the cell into an animal, or the like.

Various devices and materials may used in connection with invetion see exemplary methods and compositions provided in U.S. Pat. Nos. 6,445,938; 6,430,424; 6,428,811; 6,424,851; 6,424,849; 6,424,848; 6,421,548; 6,362,144; 6,040,292; 5,993,799; 5,741,211, Each of which is incorporated herein by reference.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Culture and Nuclear Extract Preparation

Monolayer cultures of the pancreatic islet β-cell (βTC-3 (Efrat, 1988) and MIN-6 (Miyazaki, 1990)) and α-cell (αTC6; Hamaguchi, 1990) lines were grown under conditions described previously. Non-islet cell, Hela cells were maintained in Dulbecco's MEM (GIBCO BRL, Gaithersburg, Md.) supplemented with 10% heat-inactivated fetal calf serum, 100 units/ml penicillin, 100 mg/ml streptomycin at 37° C. in a humidified atmosphere of 5% CO2 and 95% air. Human islets were provided by the Juvenile Diabetes Foundation International Human Islet Distribution Program at Washington University and were cultured in CMRL medium (GIBCO BRL, Gaithersburg, Md.) with 10% heat-inactivated fetal calf serum. Human islet and those cell line's nuclear extracts were prepared by the procedure described by Schreiber et al, 1989, except that 1 mM phenylmethylsulfonyl fluoride was included in the high salt nuclear resuspension buffer.

2D-Gel Electrophoresis

Three hundred micrograms of βTC-3 nuclear extracts for South-Western blotting and Western blotting were dialyzed in no salt buffer (20 mM Tris-Cl pH 7.9, 1.0 mM EDTA, 1.0 mM EGTA, 10% glycerol, 1.0 mM DTT and 1.0 mM PMSF) and precipitated with 20% trichloroacetic acid (TCA). Then, the pellets were resuspended in 300 μl of rehydration buffer (8.0 M urea, 4.0% CHAPS, 2.0 mM tributyl phosphine and 0.2% (w/y) Biolytes 3/10) for 17 cm immobilized pH gradient (IPG; Bio-Rad Laboratories, Hercules) gels. Separation on the first dimension was performed with IPG strip using a linear pH gradient from 3-10. After focusing of 17 cm IPG strips for 85,000 V-h, stripes were equilibrated for 10 min in equilibration buffer (6.0 M urea, 0.375 M Tris-Cl pH 8.8, 2.0% SDS and 20% glycerol) with 2.0% (w/v) DTT and for additional 10 min in equilibration buffer with 2.5% (w/v) iodoacetamide. After equilibration, the strips were cut into 6 cm and loaded on a 10% SDS-PAGE gel. Gels were run at 200 V for 50 min and were either blotted, stained, or cut for elution of proteins.

South-Western Blotting

After 2D-gel electrophoresis, proteins were transferred onto nitrocellulose membranes at 200 mA for 3 hours in TG buffer which was made of 20 mM Tris-Cl pH 8.0 and 150 mM glycine. Transferred proteins were renatured on the membrane with renature buffer (10 mM Tris-Cl pH 7.4, 1.0 mM EDTA, 100 mM NaCl, 10% Glycerol, 2.0 mM DTT, 1 mM PMSF, 1.0% Triton and 1.0 mM sodium orthovanadate) for 30 min at room temperature with gentle rocking. The membranes were left in binding buffer (10 mM Tris-Cl pH 7.4, 1.0 mM EDTA, 100 mM NaCl, 10% Glycerol, 2.0 mM DTT, 1 mM PMSF, 1.0% Triton and 10.0 mM sodium orthovanadate) with βTC6 or MIN6 cell nuclear protein (500 μg/ml binding buffer ) on rocking shaker for 30 min at room temperature. Then, membranes were rinsed by hybridization buffer (10 mM Tris-Cl pH 7.4, 1.0 mM EDTA, 100 mM NaCl, 10% Glycerol, 2.0 mM DTT, 1 mM PMSF, 1.0 mM sodium orthovanadate) for 5 min at 4° C. Hybridization was performed after addition of poly(dI-dC) (5 μg/ml hybridization buffer) and a double-stranded ³²P-labeled probe (5 ng, 5×10⁵ cpm/ml hybridization buffer) containing C1/RIPE3b1 (−126 TGGAAACTGCAGCTTCAGCCCCTCTG-101)(SEQ ID NO:5) element sequences from the rat insulin II gene. After the rinse of membranes 3 times for 10 min in hybridization buffer and being briefly dried on a Whatmann paper, the blot was exposed to the films.

Elution and Tenature of RIPE3b1 Activator from Polyacrylamide Gel

After rinsing of the SDS-PAGE gels in Tris-Glycine buffer (20 mM Tris-Cl pH 8.0 and 150 mM glycine) for 20 min, small pieces of gel were cut out precisely. The gel slices were grounded with a hand held homogenizer and proteins were eluted in renature buffer (10 mM Tris-Cl pH 7.4, 1.0 mM EDTA, 100 mM NaCl, 10% Glycerol, 2.0 mM DTT, 1 mM PMSF, 1.0% Triton, 100 μg/ml BSA and 1.0 mM sodium orthovanadate) on rotating wheel for 4 hr at 4° C. After the centrifuged supernatants which include eluted proteins were stored at −70° C.

Western Blotting Analysis

Nuclear extracts were separated by SDS-PAGE (10% resolving gel) at 200 V for 50 min. For 2D Western blotting, before SDS-PAGE, protein samples were isoelectric focused on IPG strip as mentioned before. Proteins were transferred onto nitrocellulose membranes at 35 V for 1 hour in transfer buffer (25 mM Bicine, 25 mM Bis-Tris, 1.0 mM EDTA, 50 nM Chlorobutanol). After blocking the membranes for 1-2 hours at room temperature in PBST buffer (PBS with 0.05% Tween-20) containing 5% skim milk, the membranes were incubated at room temperature for 60 min in PBST buffer with 5% skim milk containing 1:10,000 dilution of anti-Maf polyclonal antibody ((αMaf; 2.0 μg/μl), 1:25,000 dilution of anti-MafB polyclonal antibody (αMafB; 2.0 μg/μl) or 1:2000 dilution of anti-c-Maf polyclonal antibody (αc-Maf; 2.0 μg/μl). All Maf antibodies were from Santa Cruz Biotechnology (Santa Cruz, Calif.). Membranes were then washed three times for 10 min each time in PBST and incubated for 60 min at room temperature in PBST with 5% skim milk containing 1:10,000 dilution of goat anti-rabbit IgG or donkey anti-goat IgG antibody coupled with horseradish peroxidase. After washing the membrane three times for 10 min each time with PBST, immunoreactive bands were visualized by incubation with Lumi-Light Western Blotting Substrate (Roche, Mannheim, Germany) and exposed to the film.

Electrophoreic Mobility Shift Assays

Adequate amounts of sample proteins, such as islet, βTC-3, and Maf over-expressed Hela cell nuclear extracts and eluted proteins from gels, were preincubated in mobility shift buffer (10 mM Tris-Cl pH 7.4, 100 mM NaCl, 2 mM DTT, 10% (v/v) glycerol) with 1 μg poly(dI-dC) for 15 min at 4° C. For elution of a protein from the gel, poly(dI-dC) was not added. When antibodies were required, 0.4 μg of αMaf, 0.04 μg of αMafB or 0.4 μg of αc-Maf were put into mobility shift buffer 15 minutes prior to addition of the probe. The binding reaction was initiated by addition of a double-stranded ³²P-labeled C1/RIPE3b1 probe (5′-TGGAAACTGCAGCTTCAGCCCCTCTG-3′ (SEQ ID NO:6); 1 ng, 1×10⁵ cpm). To see the specific bindings, 10 times excess amount of −111/−108 mutant competitor (5′-TGGAAACTGCAGCTTACTACCCTCTG-3′)(SEQ ID NO:7) or wild type competitor was added in mobility shift buffer before adding probe. Each of the binding reactions were incubated for 30 min at 4° C., and then the complexes resolved by electrophoresis through a 6% non-denaturing polyacrylamide gel using high ionic strength polyacrylamide gel electrophoresis (PAGE) conditions.

Purification of RIPE3b1 Activator

The purification of RIPE3b1 activator were achieved using modified DNA affinity chromatography, oligonucleotide trapping method (Gadgil, 2001). For this method, double strand insulin DNA from −126/−101 bp (5′-TGGAAACTGCAGCTTCAGCCCCTCTG-3′)(SEQ ID NO:8) which involve C1/RIPE3b1 element and contain a GTGTGTGTGT single oligonucleotide tail, C1/RIPE3b1(GT)5, was prepared. At the first step of purification, βTC-3 nuclear extract dialyzed in 150 mM binding buffer (10 mM Tris-Cl pH 7.4, 1.0 mM EDTA, 10% glycerol, 150 mM NaCl, 1 mM DTT and 1 mM PMSF) was mixed with C1/RIPE3b1(GT)5 (60 pmol/mg nuclear protein), poly(dI-dC) (20 μg/mg nuclear protein) and protease inhibitor cocktail (1 tablet/50 ml sample) (Roche, Mannheim, Germany), and allowed to rock on a shaker for 15 min at 4° C. to make RIPE3b1 activator-DNA complex. The RIPE3b1 activator binding complex was then applied to a sepharose column containing the cross-linked single-stranded ACACACACAC oligonucleotide ((AC)5) column. The column was then washed with 150 mM binding buffer at least 3 times volume of applied sample. The retained RIPE3b1 activator was eluted from the DNA trapped on (AC)5 resin by NaCl gradient elution, from 0.15 to 1.5 M NaCl. For this first affinity column purification, 600 mg of βTC-3 nuclear extract was used. As a second step, after dialyzing the first affinity protein in 150 mM binding buffer, it was applied to (AC)5 column twice to remove contaminating proteins which bind (AC)5 resin. The flow through from (AC)5 column was used for the next step of purification. To decrease the non-specific binding protein, an oligo DNA from PDX-1's −2106/−2083 enhancer region (5′-TCTTTTTGCAAAGCACAGCAAAAA-3′)(SEQ ID NO:9) which is another RIPE3b1 activator binding element (in submit) was used instead of C1/RIPE3b1 element. A second oligonucleotide trapping method was performed as well as first DNA affinity purification. In each step, SDS-PAGE followed by silver stain (Bio-Rad; Laboratories, Hercules, Calif.) and gel-shift assay was performed to confirm the RIPE3b1 purification. After the 2nd affinity purification, protein was applied to 2D electrophoresis and the gel was Coomassie stained by using Colloidal Blue Staining Kit (Invitrogen, Carlsbad, Calif.). Stained spots on 46 kDa at pH7 and 4.5 were cut out and trypsine digestions were performed.

Cloning of the Mouse L-Maf

The peptide sequence from MS matched with chicken L-Maf and two uncharacterized mouse DNA sequence. By the comparison of chicken L-Maf and these mouse DNA, primer set to amplify mouse L-Maf coding sequence was designed (5′-ATGGCCGCGGAGCTGGCGATG-3′(SEQ ID NO:10) and 5′-TCAGAAAGAAGTCGGGT-3′)(SEQ ID NO: 1), and performed RT-PCR with RNA from βTC-3 cell. RT-PCR method was performed with One-Step RT-PCR kit (CLONTECH, Palo Alto, Calif.) initiated with oligo dT RT-primer. To confirm the splicing site, mouse genomic DNA (CLONTECH, Palo Alto, Calif.) was also used as template. Then, amplified DNA was cloned in pcDNA3.1/Zeo(+) (Invitrogen, San Diego, Calif.) and sequenced.

RNA Isolation and Evaluation of Maf Gene Expression

Mouse pancreatic islets were isolated from B6D2 mice. The pancreas was washed with cold Hank's balanced salt solution (HBSS; GIBCO BRL, Gaithersburg, Md.) and diced into 1 mm pieces by scissors in cold HBSS. Pancreatic tissue was then placed into a glass tube with 5 ml cold HBSS. Twelve milligrams of collagenase P (Roche, Indianapolis, Ind.) were added and submerged in a shaker water bath at 37° C. with rapidly shaking for 12 minutes. The tube was centrifuged 1000 about 30 seconds, the supernatant was discarded and the pellet was resuspended in cold HBSS. Washing was continued until the supernatant was clean. The islets were then, poured into an untreated 100 mm petri dish with islet medium (11 mM glucose, 10% Fetal Bovine Serum and 100 U/ml penicillin/streptomycin in RPMI 1640; GIBCO BRL, Gaithersburg, Md.). Using a micro pipette and dissecting microscope, 500 islets were individually picked up and homogenized in potter type homogenizer to isolate RNA. Total RNA from mouse islets, βTC-3 and αTC6 was isolated by using TRIZOL Reagent (GIBCO BRL, Gaithersburg, Md.), and less than 50 μg of RNA was treated with 10 unit of DNaseI in 10 mM Tris-Cl, pH 8.4, 50 mM KCl, 1.5 mM MgC and 0.001% gelatin at 37° C. for 30 min to remove contaminating DNA. After the DNaseI treatment, RNA was purified by TRIZOL Reagent once again.

RT-PCR method was performed with One-Step RT-PCR kit (CLONTECH, Palo Alto, Calif.). Reverse transcription used 100 ng mouse islet, 500 ng βTC-3 or TC6 total RNAs were primed with 20 pmol oligo(dT) primer at 50° C. for 60 minutes, then PCR-amplified for 32 cycles of 94° C./45 seconds, 62 (L-Maf), 59 (MafB), 58 (c-Maf) ° C./45 seconds, 72° C./1 minute with 20 pmol of each primer. Oligonucleotide primers and size of the amplified products for the different genes assayed were as follows. Mouse L-Maf, 5′-AGGCTTTCCGGGGTCAGAGT-3′ (SEQ ID NO:12) and 5′-TGGAGCTGGCACTTCTCGCT-3′ (SEQ ID NO:13), 403 bp; mouse MafB, 5′-CAACAGCTACCCACTAGCCA-3′ (SEQ ID NO:14) and 5′-GGCGAGTTTCTCGCACTTGA-3′ (SEQ ID NO:15), 366 bp; mouse c-Maf, 5′-GTGCAGCAGAGACACGTCCT-3′ (SEQ ID NO:16) and 5′-CAACTAGCAAGCCCACTC-3′ (SEQ ID NO:17), 272 bp. As a control, 100 ng mouse genomic DNA and same amount of mouse islet, βTC-3 or αTC6 RNA as RT-PCR were used for PCR with the same PCR kit without reverse transcriptase. The PCR products were confirmed by diagnostic restriction-enzyme digestion. Amplified products were electrophoresed through a 1.5% agarose gel in TAE buffer and visualized by ethidium bromide staining.

Maf Expression Plasmids and Reporter Gene Analysis

The Maf expression plasmids was constructed using PCR-mediated strategy. The mouse L-Maf cDNA contained all coding sequences was isolated by RT-PCR method using total RNA from βTC-3 cells, and the mouse MafB and c-Maf coding sequence were obtained by PCR from mouse genomic DNA (CLONTECH, Palo Alto, Calif.), and mouse c-Maf expression plasmid, RcRSVcMaf, respectively. After the sequence fidelity was confirmed, those cDNAs were cloned into the HINDIII and XbaI site of the expression vector pcDNA3.1/Zeo(+) (Invitrogen, San Diego, Calif.). L-Maf cloned pcDNA3.1/Zeo(+) was used as a template for site directed mutagenesis (QuikChange™Site-Directed Mutagenesis Kit; STRATAGENE, La Jolla, Calif.) to substitute serine 14 and/or serine 65 with alanine (S14A, S65A, and S14A/S65A). The following oligonucleotides were used: for S14 and for S65 [Benkhelifa, 2001]. For the gene transfection, Hela cells were replated in six-well tissue culture plates 24 h before transfection. One microgram of reporter and each Maf expression plasmids were co-transfected with 2 ng of internal control plasmid, pRL-CMV (Promega, Madison, Wis.) which contains Renilla luciferase gene promoted by CMV immediate-early enhancer/promoter region. These plasmids were transfected by the lipofection method using lipofectamine TM2000 reagent (Invitrogen, Carlsbad, Calif.) under the conditions recommended by the manufacturer. To evaluate Mafs effects on Insulin promoter activity, two reporter constructs were used. One was −238 WT LUC reporter plasmid (Robinson, 1994) which contain rat insulin II gene sequence from −238 to +2 bp upstream of firefly luciferase. Another one was −238 MT LUC which was generated from −238 WT LUC by putting mutation on −111/−108 within insulin's C1/RIPE3b1 element by using the QuikChange™Site-Directed Mutagenesis Kit (STRATAGENE, La Jolla, Calif.). Oligonucleotides were used for −238 MT LUC. Transfected cells were incubated for 48 hours and harvested. The preparation of cell extracts and measurement of luciferase activities were carried out using the Dual-Luciferase reporter assay system according to the recommendation of the manufacturer (Promega, Madison, Wis.). The assays for firefly luciferase and Renilla luciferase activity were performed sequentially using one reaction tube in a luminometer with two injectors. Changes in firefly luciferase activity were calculated and plotted after normalization with change in renilla luciferase activity in the same sample. Then Mafs effect on insulin promoter was shown as relative luciferase activity after normalized by no Maf transfected insulin promoter activity. Each experiment was repeated at least 3 times.

ChIP Assay

βTC-3 cells (˜0.5×10⁸ to 1.0×10⁸) were formaldehyde cross-linked, and the sonicated protein-DNA complexes were isolated under conditions described previously (Gerrish, 2000). αMaf (10 μg), normal rabbit immunoglobulin G (IgG; 10 μg; Santa Cruz Biotechnology, Santa Cruz,Calif.) antibody, or no antibody was added to the sonicated chromatin, followed by incubation for 1 h at 4° C. Antibody-protein-DNA complexes were isolated by incubation with A/G-agarose (Santa Cruz Biotechnology, Santa Cruz,Calif.). PCR was performed on one-tenth of the purified, immunoprecipitated DNA by using Ready-to-Go PCR beads (Amersham Pharmacia Biotech, Piscataway, N.J.) and 15 pmol of each primer. The primers used for amplification of mouse insulin promoter region were −378 (5′-GGAACTGTGAAACAGTCCAAGG-3′ SEQ ID NO:18) and −46 (5′-CCCCCTGGACTTTGCTGTTTG)(SEQ ID NO:19) These oligonucleotides amplify both the mouse insulin I and II. The primers used for amplification of mouse PEPCK (phosphoenolpyruvate carboxykinase) were −434 (5′-GAGTGACACCTCACAGCTGTGG-3′ SEQ ID NO:20) and −96 (5′-GGCAGGCCTTTGGATCATAGCC-3′ SEQ ID NO:21). PCR cycling parameters were 1 cycle of 95° C./2 minutes and 28 cycle of 95° C./30 seconds, 61° C./30 seconds, 72° C./30 seconds. The PCR products were confirmed by sequencing. Amplified products were electrophoresed through a 1.5% agarose gel in TAE buffer and visualized by ethidium bromide staining.

Immunohistochimistry

The Vectastain ABC (Vector Laboratories) and Histomouse SP (Zymed Lab-SA System; Zymed Laboratories, Inc.,) kits were used for Mafs, insulin and glucagon immunohistochemical staining.

### Example 2

### Results

RIPE3b1 Activator has Two Isoelectric Points

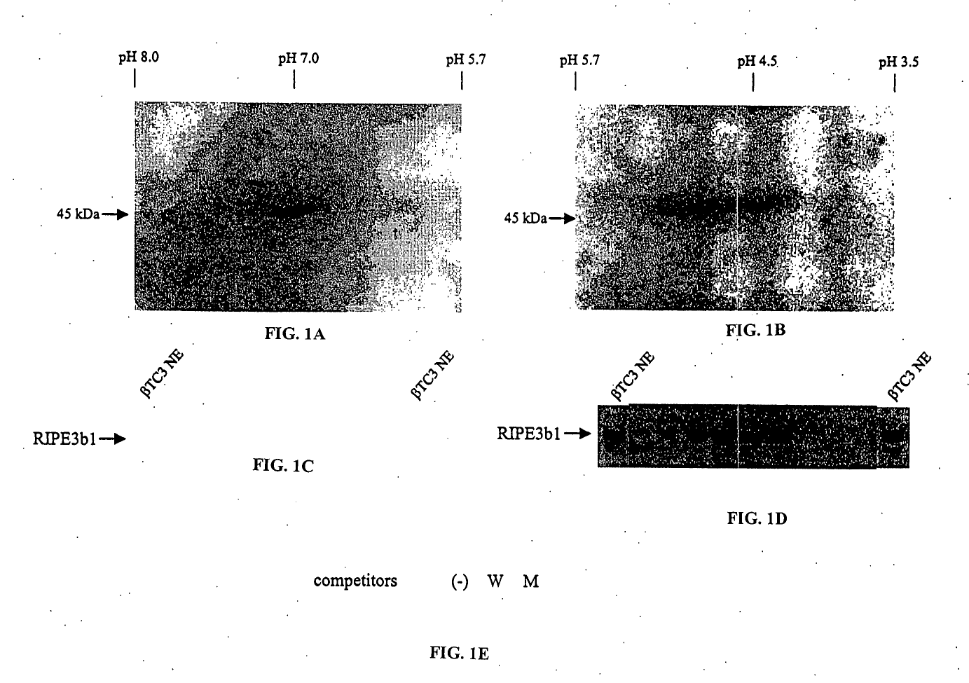

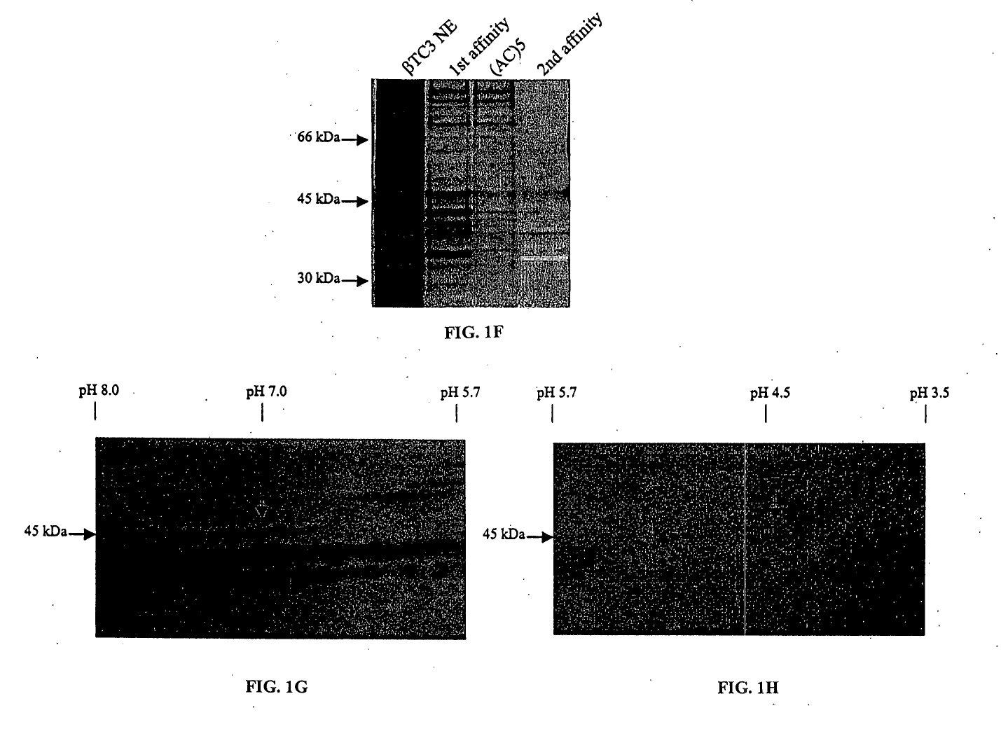

For the isolation of RIPE3b1 activator, multiple techniques were required to facilitate the ultimate purification of the protein. At first, a South-Western blotting strategy was established to detect RIPE3b1 binding protein using insulin C1/RIPE3b1 element as a probe. The 46 kDa RIPE3b1 DNA-binding protein(s) was demonstrated to be highly enriched in islet β cell lines, βTC-3 and MIN6 cells (data not shown). In addition, its isoelectric points were found to be roughly pH7.0 and 4.5 upon analysis of βTC-3 nuclear proteins separated by 2D gel electrophoresis (FIG. 1A-B). Its binding specificity to C1/RIPE3b1 probe was confirmed by competition analysis using mutated C1 RIPE3b1 probe (data not shown). The RIPE3b1 DNA binding activity was also detected in the eluted protein(s) from those spots on 2D gel indicated by South-Western blotting (FIG. 1C-D). Competition assay on gel-shift analysis showed these eluted protein bind C1/RIPE3b1 probe specifically (FIG. 1E). Collectively, South-Western blotting analysis clearly detected the position of RIPE3b1 activator on 2D gel, and was utilized as one step of RIPE3b1 purification.

Purification of RIPE3b1 Binding Protein

As the first step of purification, modified DNA affinity chromatography, oligonucleotide trapping method was used (Gadgil, 2001). This method has given both a higher yield and significantly purer protein than usual DNA affinity chromatography matrix (data not shown). RIPE3b1 activator, detected by gel-shift analysis, was eluted between 500 and 700 mM NaCl from chromatography column (data not shown). At the DNA affinity purification step, about 40% of RIPE3b1 activity was retained, and in two applications of the first affinity protein into (AC)₅ column, little RIPE3b1 activity was lost. To evaluate the result of purification, each step of purified sample which possess the same RIPE3b1 activity was run on an SDS-PAGE 10% gel and silver-stained. Although several bands were still detected in the second affinity protein, the 46 kDa protein was mainly enriched through the purification steps (FIG. 1F). It was also elucidated that the second affinity protein has strong RIPE3b1 activity by competition on gel-shift assay (data not shown). Since MS analysis used 46 kDa band of second affinity protein showed that it contain significant amount of AU-rich element RNA-binding protein, AUF-1 (Zhang W, 1993) (data not shown), the inventors performed RIPE3b1 gel-shift assay with AUF-1 antibody. As AUF-1 antibody did not recognize RIPE3b1 complex at all (data not shown), concluded AUF1 is a contaminationg protein. That conclusion was consistent with the findings that AUF1 binds (AC)5 single strand oligonucleotide, from the results of oligonucleotide trapping method performed without adding C1/RIPE3b1 probe. 2D electrophoresis was performed after the second affinity purification. As South-Western blotting analysis indicated 2 protein spots at pH7.0 and 4.5 on 46 kDa were observed on Coomassie stained 2D gel (FIG. 1G -1H).

Identification and Cloning of RIPE3b1 Activator

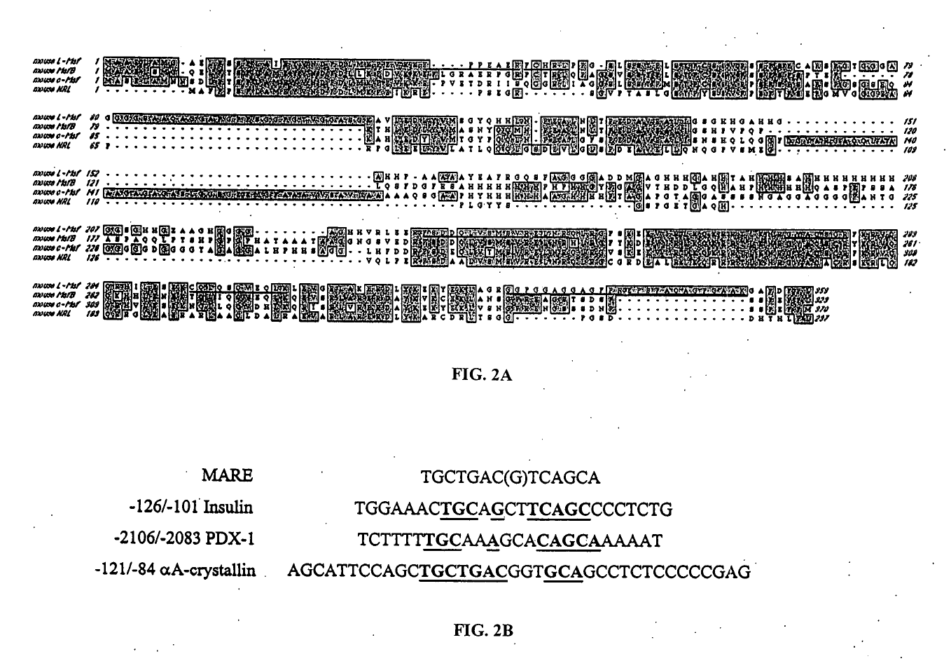

The 46 kDa protein at pH7.0 and 4.5 were subjected to HPLC-MS/MS (or MALDI-TOF MS) in Vanderbilt Mass Spectroscopy facility. The peptide patterns obtained (FIG. 2) were then analyzed using Protein-Prospector, MS-Fit and PeptIdent programs, resulting in several matches with an uncharacterized mouse cDNA and chicken basic leucine zipper transcription (bZIP) factor L-Maf (GenBank accession No. AF034570). Since these unknown mouse DNAs and chicken L-Maf DNA sequence showed high similarity when aligned, it was supposed that the RIPE3b1 activator might be mouse L-Maf. Although these database gave only 5′ and 3′ end of mouse L-maf, a designed mouse primer set from the comparison with chicken L-Maf was word to amplify the whole coding region of mouse L-Maf by RT-PCR. In addition, PCR with mouse genomic DNA produced the same length of DNA with RT-PCR, this indicated that the mouse L-Maf has no intron as other mouse large Mafs (data not shown). Mouse L-Maf amino acid sequence is shown in FIG. 2A with other mouse large Maf family aligned. As indicated by underline, MS detected 8 N— and C-terminal peptides of mouse L-Maf and most peptides had mouse L-Maf specific sequence, therefore, this indicates, that isolated protein from βTC-3 cell using RIPE3b1 DNA afinity purification was mouse L-Maf protein.

Large Mafs can Bind C1/RIPE3b1 Cis-Element Specifically.

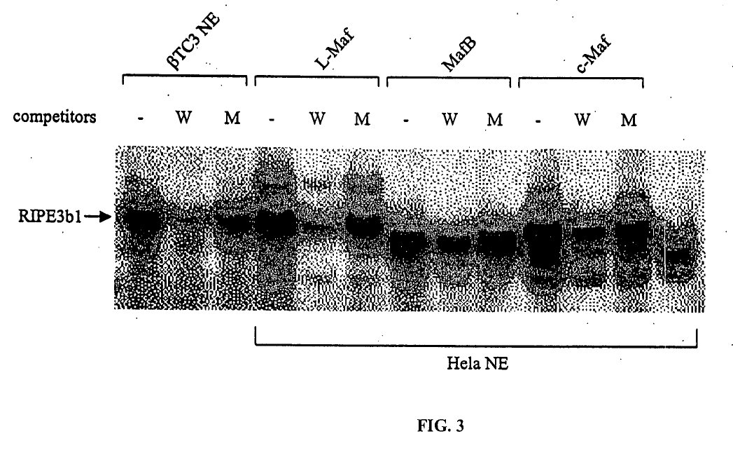

On gel-shift analysis, large Mafs which were over-expressed in Hela and βTC-3 indicated the same migration (data not shown), and since Hela cell nuclear extract has no protein which show specific binding to C1/RIPE3b1 probe on gel-shift assay (data not shown), over-expressed Maf proteins in Hela cell were used as a sample for the following experiments. As expected because of its similarity between C1/RIPE3b1 insulin cis-element and MAREs (FIG. 2B), L-Maf, MafB and c-Maf bound C1/RIPE3b1 probe (FIG. 3). Binding of L-Maf, MafB and c-Maf to C1/RIPE3b1 probe were competed by wild type competitor but not by −108/111 mutant (FIG. 3). Namely, these large Maf bound C1/RIPE3b1 insulin cis-element selectively. The another competitor, mouse αA-crystallin's −121/−84 cis-element (FIG. 2B) which is well known as Maf binding site (Brian, 2000), competed binding of RIPE3b1 activator in βTC-3 nuclear extract, MafA, MafB and c-Maf to C1/RIPE3b1 element to the same degree as C1/RIPE3b1 wild competitor (data not shown). These results suggested that C1/RIPE3b1 insulin cis-element strongly binds to these Mafs at least in same degree as αA-crystallin's MARE.

Maf Western Blotting with Anti-Maf Antibodies

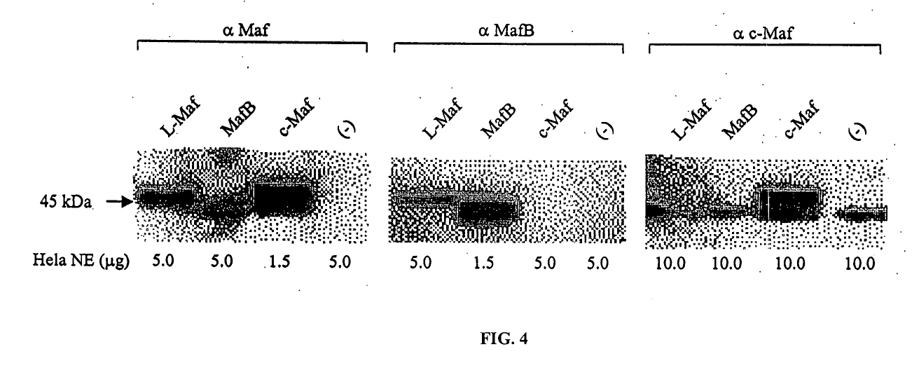

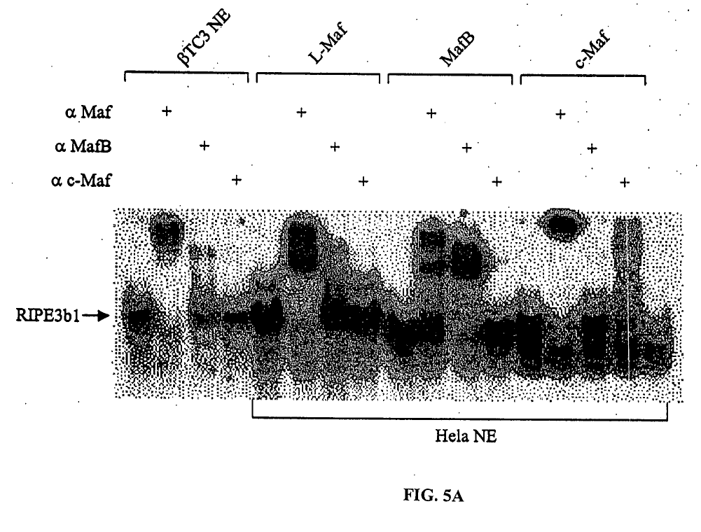

Large Mafs specific and strong binding to C1/RIPE3b1 indicated which Maf may be a RIPE3b1 activator. To address the question, the specificity of anti-Maf antibodies were evaluated by Western blotting analysis using L-Maf, MafB ot c-Maf over-expressed Hela cell nuclear extracts. Since αMaf correspond to amino acids 19-171 of mouse c-Maf, it recognize not only c-Maf but also mouse L-Maf very well, and it also detected MafB weakly on Western blotting (FIG. 4). On the other hand, αMafB principally recognized MafB, and poorly L-Maf. In contrast, αc-Maf appeared to detect only c-Maf, although its sensitivity was weak. FIG. 4 also showed over-expressed L-Maf and c-Maf were nearly 46 kDa and MafB is around 45 kDa protein in Hela cell as well as in βTC3 and αTC6, in addition, each of MafB and c-Maf revealed two close bands. These findings, that Molecular weights of large Mafs are very close to each other and antibody specificity and sensitivity seemed to be quite different, made it difficult to decide which Maf is dominant in islet β-cell.

αMaf Tecognize RIPE3b1 Complex

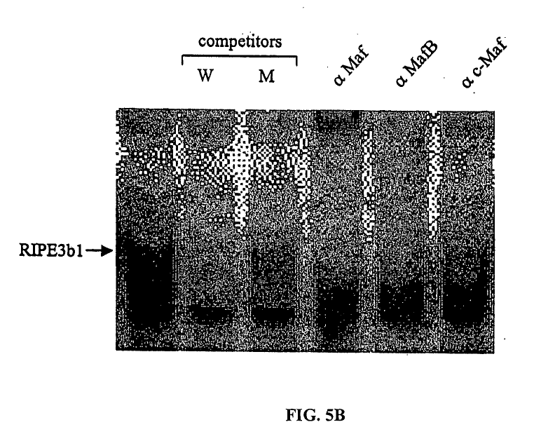

To decide if RIPE3b1 is Maf, gel-shift assay was performed with anti-Maf antibodies. The αMaf entirely super-shifted RIPE3b1 complex in βTC-3 nuclear extract as well as L-Maf and c-Maf complex, while it weakly super-shifted MafB complex (FIG. 5A). These results certified RIPE3b1 activator belongs to large Maf family and αMaf recognizes L-Maf, c-Maf strongly and MafB weakly. On the other hand, αc-Maf did not recognize RIPE3b1, L-Maf and MafB complex at all, while c-Maf is super-shifted (FIG. 5A). Concerning about αMafB, it super-shifted MafB completely and recognized MafA and RIPE3b1 complex very weakly (FIG. 5A), furthermore, MafB, whose migration in Hela and βTC-3 cells is same on gel-shift assay, migrated sgnificantly faster than RIPE3b1 complex. These antibodies effects on RIPE3b1 complex in βTC3 nuclear extract were same as that in islet nuclear extract (FIG. 5B). These results suggest that c-Maf and MafB are not major components of the RIPE3b1 activator.

Maf's Effects on Insulin Promoter

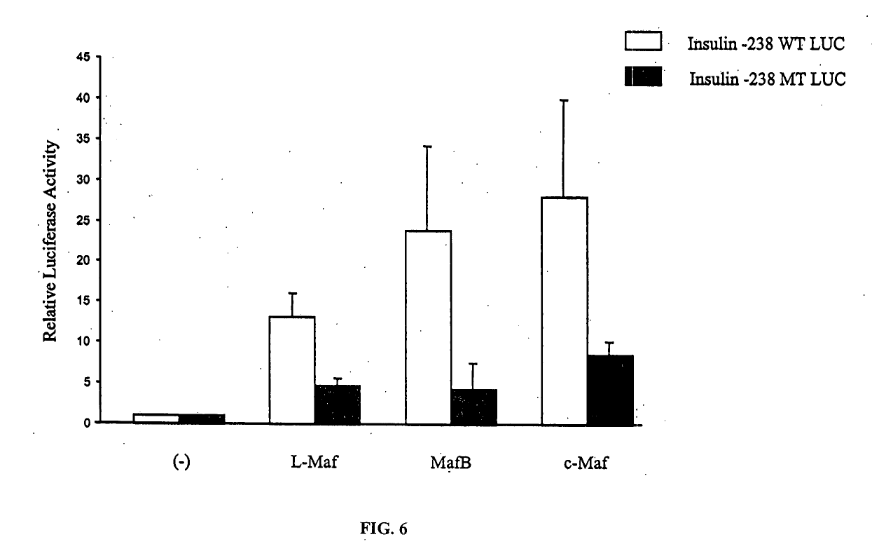

To evaluate the ability of Mafs to activate insulin promoter, each Maf expression plasmid was cotransfected with wild (−238 WT LUC) or mutated type (−238 MT LUC) insulin promoter-driven luciferase reporter plasimids in non-islet β-cell line, Hela cells (FIG. 6A). L-Maf, MafB and c-Maf significantly activated insulin promoter, on the other hand, the mutation in −108/−111 decreased insulin promoter activity brought by these Mafs (13.2±2.8% to 4.8±0.8% for L-Maf; 23.9±10.4% to 4.3±3.3% for MafB; 28.0±12.1% to 8.5±1.7% for c-Maf). These results demonstrated large Mafs activate insulin promoter through C1/RIPE3b1 element. Similar results were observed in islet β-cell line, such as HIT-T15 cells (data not shown). Recently, it was reported that the integrity of serine 14 and serine 65 residue, which were indicated to be phosphorylated, was required for MafA (quail homolog of L-Maf) transcription activity on QR1 gene in neuroretina cells (Benkhelifa). To see if these phosphorylation sites are critical for mouse L-Maf transcription activity on insulin promoter, wild or mutant L-Maf expression vector were cotransfected with −238 WT LUC in Hela cells. However, neither S14A, S65A, nor double mutant S14A/S65A indicated significant difference from wild L-Maf on insulin promoter activity (FIG. 6B). Therefore, it was suggested that L-Maf transcription activity is regulated in target gene or cell dependent manner.

Distribution of Mafs Expression in Islet Cells

To evaluate if RIPE3b1 activator is Maf, 2D Western blotting analysis was performed with αMaf using βTC-3 nuclear extract. As FIG. 1, Maf protein was detected at pH7.0 and 4.5 on 46 kDa where is RIPE3b1 activity (FIG. 1). Another islet β cell line, MIN6 cell showed same results, while islet α cell line, αTC6 cell did not show any signals there, when the same amount of nuclear peortein were used (data not shown). Therefore, large Maf protein appeared to be RIPE3b1 activator and abundant in islet β cells.

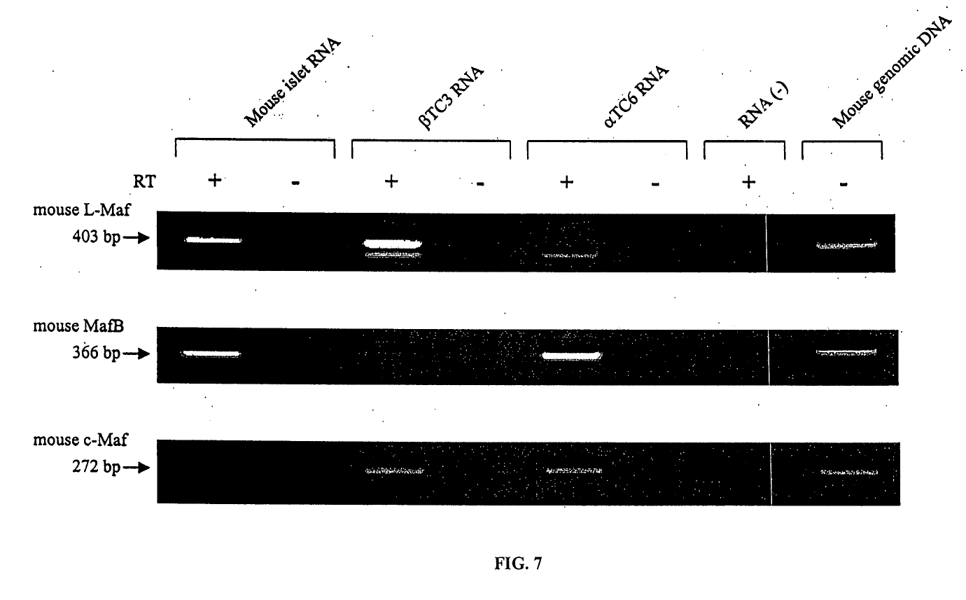

Next, to see each large Maf's expression in islet cells, RT-PCR analysis was performed with each large Maf specific primer sets. Except NRL, other 3 large Mafs, namely L-Maf, MafB and c-Maf were reproducibly amplified from mouse islet, βTC-3 and αTC6 cell RNAs by RT-PCR, while no adequate size of DNAs were amplified from these RNAs without reverse transcription. These results indicate that L-Maf, MafB and c-Maf mRNAs exist in islet, β and α cells (FIG. 7).

Maf Bind Insulin Promoter/Enhancer Region in Vivo

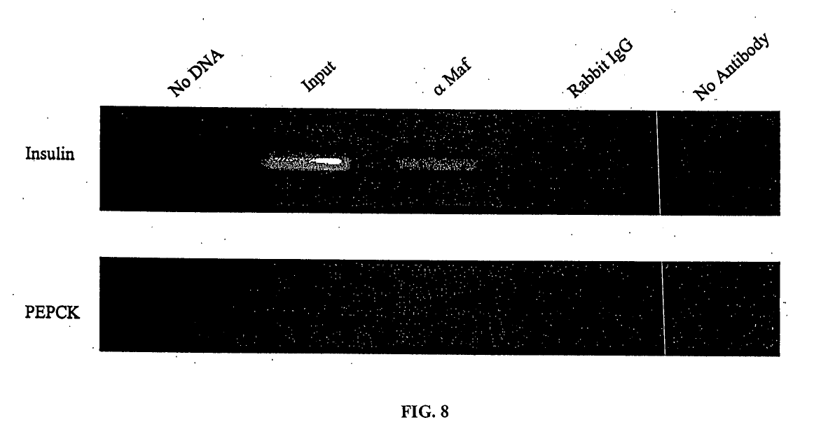

To determine whether Maf protein bind within the insulin promoter/enhancer region in vivo, ChIP assays were carried out. The αMaf immunoprecipitated insulin promoter/enhancer region from βTC-3 cells, in contrast to the rabbit IgG or the no antibody controls. In addition, αMaf did not immunoprecipitate the promoter/enhancer region of PEPCK gene, which is not transcribed in βTC-3 cells (FIG. 8). The same results were obtained with chromatin from MIN6 cells (data not shown). These results indicate large Maf binds insulin promoter/enhancer region specifically in islet β cells. Moreover, αTC6 cells were used in the same way, and αMaf immunoprecipitated glucagon promoter/enhancer region (data not shown). This finding was consistent with previous report that glucagon is transcriptionally regulated by MafA/L-Maf (Hale, 2001). The results also showed that large Maf plays an important role in islet a cell.

Maf Protein's Expression in Mouse Islet

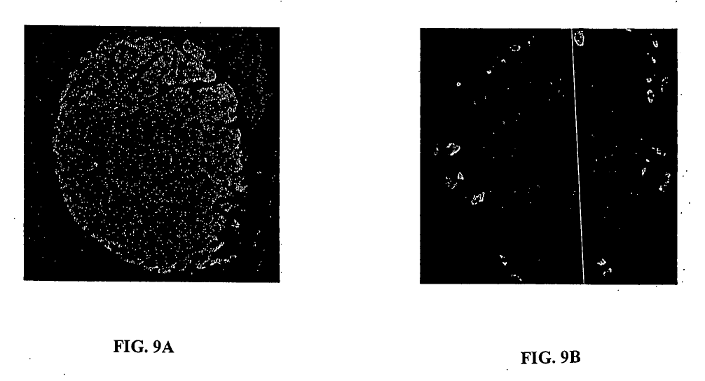

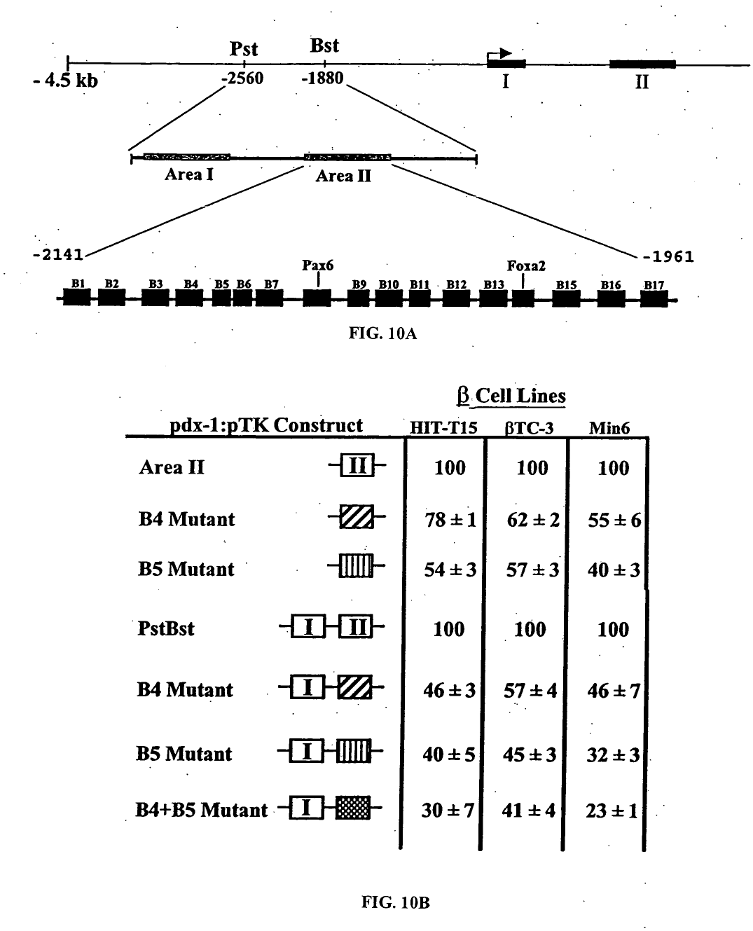

To clarify the distribution of Large Maf protein in pancreas, immunohistochemisty was performed using sections of adult mouse pancreas. Double staining with αMaf and Insulin indicated that large Maf protein looked abundant in nuclei of islet β cells but not exocrine cells (FIG. 9A). Consistently, double staining with αMaf and glucagon showed that at least L-Maf and c-Maf, which are recognized well by αMaf, are not abundant in glucagon producing cell (FIG. 9B). These results confirmed the finding that αTC6 nuclear extract has no obvious band around 46 kDa on Western blotting with αMaf while βTC3 clearly have 46 kDa protein (data not shown).

In addition, αc-Maf did not show particular staining although it is not sure whether this result means the amount of c-Maf protein is less in islet or αc-Maf's insensitivity (data not shown).

### Example 3

### Materials and Methods

Transfection Constructs

The Area II and PstBst reporter constructs were made using human (−2141/−1890 bp) and mouse (Pst/−2917bp:Bst/−1890bp) pdx-1 sequences, which were cloned directly upstream of the herpes simplex thymidine kinase (TK) promoter in a chloramphenicol acetyltransferase (CAT) expression vector, pTK(An). The block transversion and insulin C1 substitution mutants in B4/5 were constructed in Area II:pTK and PstBst:pTK using the Quick Change mutagenesis kit (Stratagene). Each construct was determined to be correct by DNA sequencing.

Cell Transfections

Monolayer cultures of pancreatic islet β (βTC-3, HIT-T15, and Min6) and non-β (NIH3T3) cell lines were maintained as described previously. The lipofectamine reagent (Gibco BRL) was used to introduce 1 μg each of Area II:pTk or PstBst:pTk and 0.5 μg pRSVLUC. The activity from the Rous sarcoma virus (RSV) enhancer-driven luciferase (LUC) plasmid served as an internal transfection control for the pdx-1:pTK constructs. LUC and CAT enzymatic assays were performed 40 to 48 h after transfection. Each experiment was carried out more than three times with at least two independently isolated DNA preparations.

Electrophoretic Mobility Shift Assays

Double-stranded Area II block 4 (B4, agcttTCTTTTTGCAAAGCACAGCAt (SEQ ID NO:22), lower case lettering corresponds to linker sequences), B5 (agcttAAAGCACAGCAAAAATATTAt (SEQ ID NO:23)) and B4/5 (agcttCTTTTTGCAAAGCACAGCAAAAAt (SEQ ID NO:24)) sequences were excised from pBluescriptKS2+, and Klenow labeled with α³²P-dATP. The Ins C1 probe spans nucleotides −126 to −101 of the rat insulin II gene and was labeled as described. Nuclear extracts were prepared as described previously, and binding reactions (20 μl total volume) conducted with 5 to 10 μg of extract protein, and labeled probe (8×10⁴ cpm) in binding buffer containing 10 mM Tris-HCl pH 7.4, 100 mM NaCl, 2 mM DTT, 1 mM EDTA, 10% glycerol, and 1 μg poly-dGdC (final concentrations). The conditions for the competition analyses were the same, except that excess of the specific competitor DNA was included in the mixture prior to addition of probe. The samples were resolved on a 6% nondenaturing polyacrylamide gel (acrylamide:bisacrylamide ratio 29:1) and run in TGE buffer (50 mM Tris, 380 mM glycine, 2 mM EDTA, pH 8.5). The gel was dried and subjected to autoradiography.

SDS-PAGE Fractionation

βTC-3 and Min6 nuclear extract (30 μg) was separated on a 10% SDS polyacrylamide gel (SDS-PAGE) and then electro-transferred onto an Immobilon polyvinylidene diflouride (PVDF) membrane (Millipore). The extract lanes were cut horizontally into 3 mm slices. The molecular weight range of each lane fraction was determined by comparison with colored Rainbow protein markers (Amersham). The proteins from each fraction were eluted as previously described and analyzed for B4/5 and Ins C1 binding activity in electrophoretic mobility shift assays.

Phosphatase Treatment

Min6 or βTC-3 nuclear extract (3-5 μg) was incubated for 10 min at 4° C. or 30° C. with and without 0.5 U of calf intestinal alkaline phophatase (CIAP; Promega) in the presence or absence of sodium orthovanadate (Na₃VO₄; 10 mM) or 10 sodium pyrophosphate (NaPPi; 10 mM) in phosphatase buffer (20 mM Tris-HCl, pH 7.4, 1 mM DTT, 0.1 mM EGTA, 2 mM MgCl₂, 1× protease inhibitor cocktail (CØmplete, Roche Diagnostics)) (10 μl total volume). The samples were analyzed for Ins C1 and B4/5 binding after addition of 10 μl of 2× gel shift binding buffer.

Anti-Phosphotyrosine Immunoprecipitation

Immunoprecipitations using anti-Tyr(P) (4G10, Upstate Biotechnology, Lake Placid, N.Y.) were performed as described previously. Briefly, SDS was added to a final concentration of 0.5% (w/v) to βTC-3 nuclear extract (100 μg protein) in a buffer containing 10 mM Tris-HCl pH 7.4, 1 mM EDTA, 10% glycerol, 1 mM Na₃VO₄, and 2 mM DTT (final concentrations), and then heated to 65° C. After diluting the SDS to 0.05%, anti-Tyr(P) or control mouse IgG was added along with protein G-Sepharose beads. The washed beads were then resuspended in 1× SDS-PAGE loading buffer and the immunoprecipitated proteins separated on a 10% SDS-polyacrylamide gel. After transfer to an Immobilon PVDF membrane, the 44-47 kDa eluted proteins were assayed for B4/5 and Ins C1 binding activity.

Chromatin Immunoprecipitation (ChIP) Assay

ChIP assays were performed with the following modifications of a described method (Gerrish, 2001; Samaras). Anti c-Maf antisera (10w1; #153, Santa Cruz Biotechnology) was incubated with sonicated formaldehyde cross-linked βTC3 chromatin. This c-Maf antiserum recognizes all mammalian members of the large Maf family (i.e., MafA, MafB, NRL, c-Maf; Santa Cruz Biotechnology and data not shown). Normal rabbit IgG (10 μg; sc-2027; Santa Cruz Biotechnology) was used as a control. The protein-DNA complexes were isolated with A/G-agarose beads (Santa Cruz Biotechnology). The PCR oligonucleotides used to detect mouse control sequences were: pdx-I Area II, −2208 5′-GGTGGGAAATCCTTCCCTCAAG-3′ (SEQ ID NO:25) and −1927 5′-CCTTAGGGATAGACCCCCTGC-3′ (SEQ ID NO:26); and Phosphoenolpyruvate carboxykinase (PCK) (−434) 5′-GAGTGACACCTCACAGCTGTGG-3′ (SEQ ID NO:27) and −96 5′-GGCAGGCCTTTGGATCATAGCC-3′ (SEQ ID NO:28). The PCR cycling parameters were 1 cycle of 95° C./2 min and 28 cycles of 95° C./30 s, 61° C./30 s, 72 C/30 s for PEPCK and 1 cycle of 95° C./2 min and 28 cycles of 95° C./30 s, 57.5° C./30 s, 72° C./30 s for Area II

### Example 4

### Results

B4 and B5 Affect Area II Activity

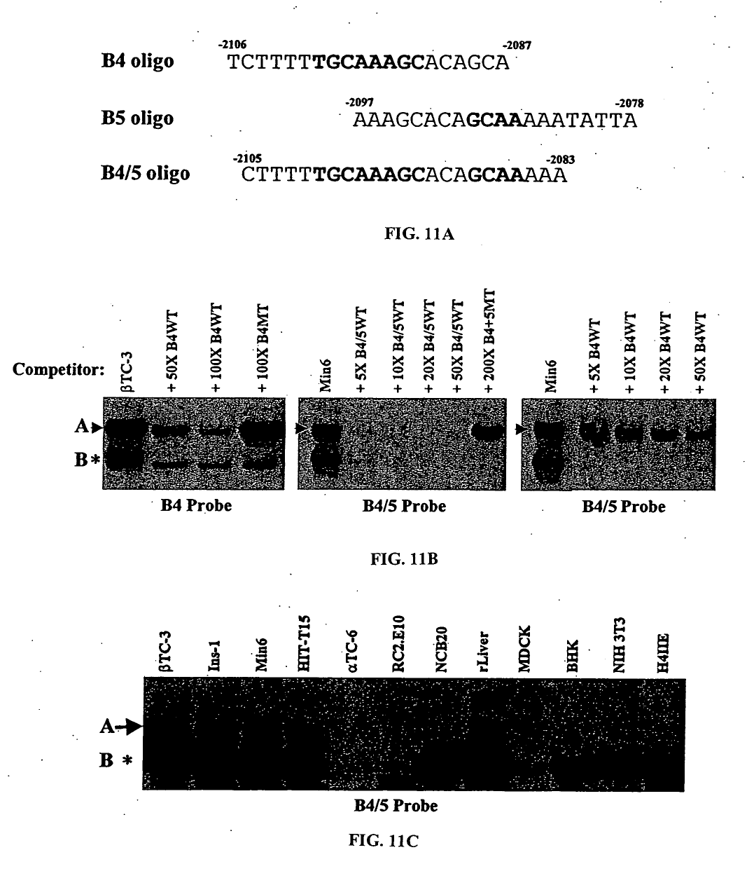

Block mutations within conserved B2 (−2131/−2115 bp), B3 (−2110/−2102bp), B4 (−2100/−2093 bp), and B5 (−2089/−2086 bp) have been shown to reduce Area II:pTK activity by in β cell lines (FIG. 11B). To further examine the significance of these elements for Area II activation, each was mutated within the mouse pdx-1 ‘PstBst’ region that spans Area I and Area II. In the context of this more active pdx-1:pTK expression construct, the B4 and B5 mutants reduced PstBst:pTK activity (FIG. 11B). To a greater extent than in Area II:pTk, and combining B4 with B5 in PstBst:pTk reduced activity further than either individual mutation (FIG. 11B). In contrast, the B2 and B3 mutants had little effect on PstBst:pTk activation (data not shown). The following experiments were designed characterize the B4 and B5 activators.

B4 and B5 Represent a Single Cis-Element that Interacts with a β Cell-Enriched Protein(s)

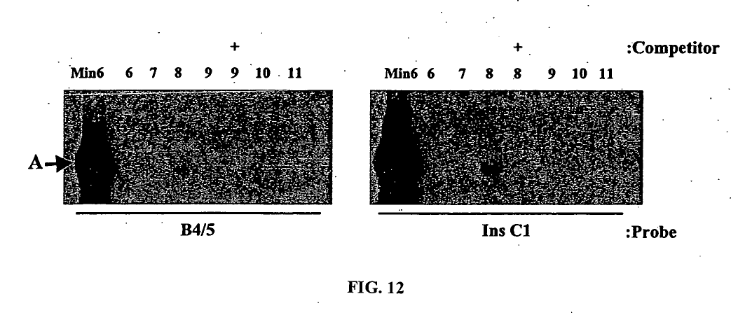

To define the factors associated with B4 and B5 mediated regulation, gel shift experiments were performed with probes spanning B4, B5, and B4+B5 (B4/5) and βTC-3 or MIN6 cell nuclear extracts (FIG. 12). Two common protein-DNA complexes were detected with the B4 and B4+B5 probes (labeled as A and B in FIG. 12B), whereas no binding was found with B5 (data not shown). The binding affinity of B4 and B4/5 for these complexes was determined with the wild type and B4+B5 double mutant site (B4+B5MT) competitors. As expected, both B4 and B4/5 reduced the levels of these complexes, although the B4/5 was roughly 20-fold more effective (FIG. 12B). In contrast, B5 did not compete for binding (data not shown), while the B4+B5MT only competed away complex B, consistent with the conclusion it is unrelated to activation (FIG. 12B). These results suggested that B4 and B5 define a single activator-binding site, which is regulated by the factor(s) found within the slower-mobility complex A.

To determine the distribution of the cellular factor(s) forming complex A, binding reactions were conducted with nuclear extracts from various islet (β: Ins-1, Min6, HIT-T15; α, αTC-6) and non-islet cell types (neuronal, RC2.E10, NCB20; liver, H4IIE normal rat liver; kidney, MDCK, BHK; fibroblast, NIH 3T3). Complex A was uniquely detected in the β cell extracts (FIG. 2B). These results suggest that the factor(s) in activator complex A is enriched in β cells.

Complex A Contains a Roughly 46 kDa Protein(s)

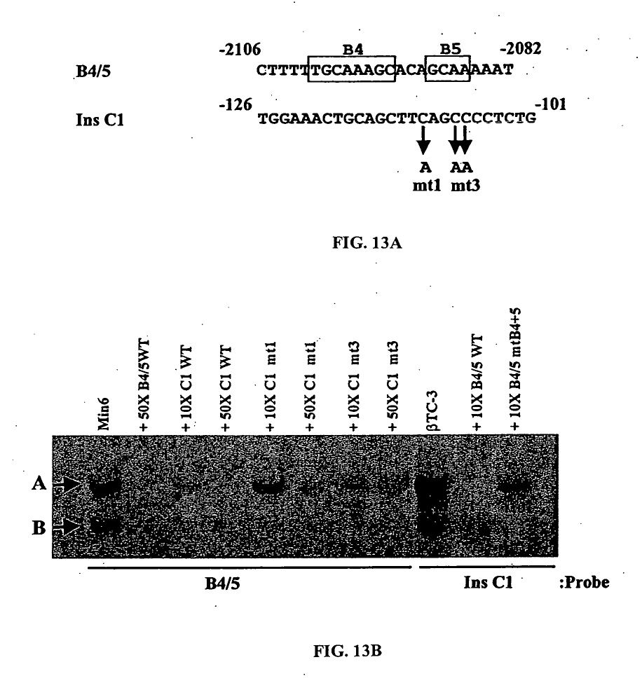

To estimate the size of the protein(s) in complex A, Min6 nuclear extracts were separated by SDS-PAGE and transferred to a PVDF membrane that was cut into slices to represent distinct molecular weights. The separated proteins were eluted from the membrane slices, renatured and tested for binding to the B4/5 probe. The binding activity in fraction 8 co-migrated with complex A found in unfractionated Min6 extracts (FIG. 13). The binding specificity of this fraction also corresponded to complex A detected in other β cell extracts (data not shown). The molecular weight range of the proteins in fractions 8 was 44 to 47 kDa. These results indicate that complex A is composed of one or more proteins of approximately 46 kDa.

The 46 kDa Complex A Protein(s) Corresponds to the Insulin C1 Activator, RIPE3b1

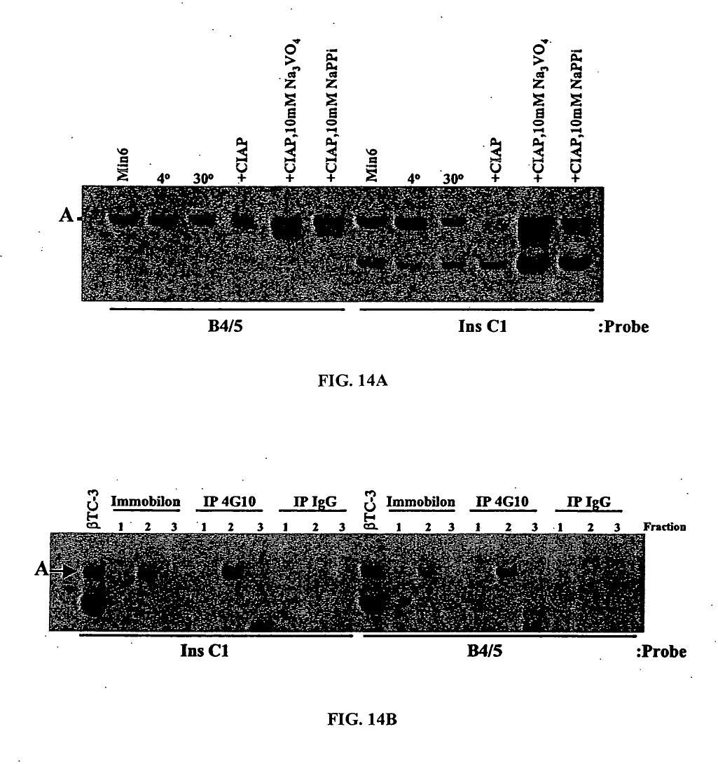

Because the RIPE3b1 protein(s) that binds to and activates the insulin C1 control element has the same cell-restricted distribution and molecular size (see FIG. 13) the binding properties of B4/5 were compared to insulin C1 (termed Ins C1) (FIG. 14). Both Ins C1 and B4/5 competed effectively for complex A binding when either B4/5 or Ins C1 were used as probes (FIG. 14B). Mutants in B4/5 or Ins C1 that either modestly (i.e. Ins C1mt1) or profoundly (i.e. Ins C1mt3, B4+B5MT (FIG. 12B)) affected RIPE3b1 or complex A binding yielded competition patterns consistent with each element binding the same factor(s) (FIG. 14B).

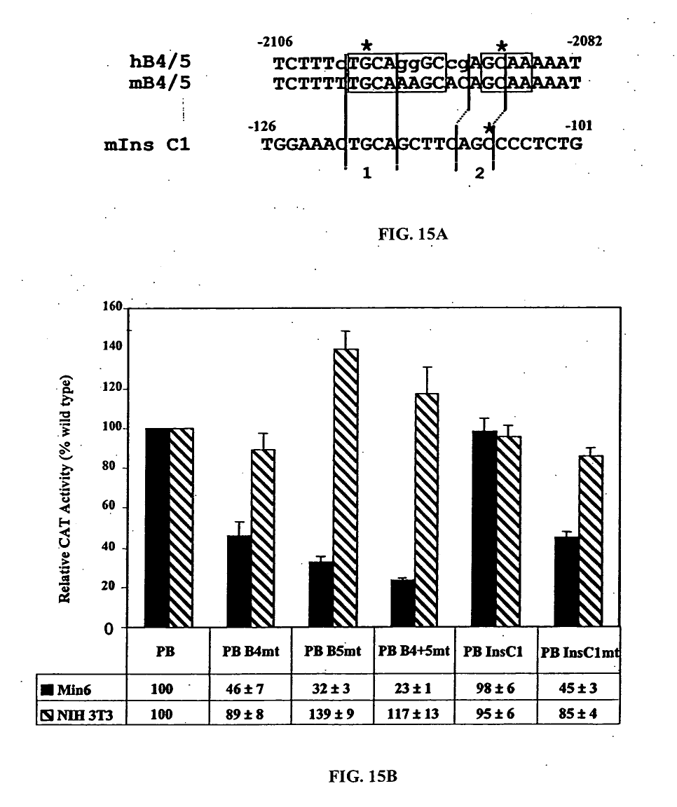

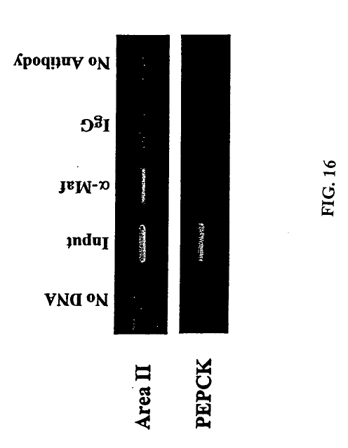

RIPE3b1 binding activity is inhibited by the actions of a tyrosine phosphatase. To test if complex A formation on B4/5 is also regulated in this manner, Min6 nuclear extracts were incubated in the presence or absence of calf intestinal alkaline phosphatase (CLAP) and a general (sodium pyrophosphate (NaPPi)) or phosphotyrosine specific (sodium orthovanadate (Na₃VO₄)) phosphatase inhibitor. B4/5 and InsC1 binding activity were monitored in the treated extracts. The binding characteristics of complex A were affected in exactly the same manner with both probes (FIG. 15A). Complex A mobility was shifted upon incubating the β nuclear extract at 30° C. with both the B4/5 and C1 probes, presumably due to the actions of an endogenous tyrosine phosphatase. CIAP treatment reduced binding to each probe, an affect blocked by addition of NaPPi or Na₃VO₄ (FIG. 1 5A). In addition, the C1 and B4/5 binding 46 kDa fraction immunoprecipated from β cell nuclear extracts using the anti-phosphotyrosine immunospecific monoclonal antibody, 4G10, was found after separation of the βTC-3 precipitate by SDS-PAGE (compare the 4G10 to IgG lanes in FIG. 15B). Collectively, these results strongly suggest that RIPE3b1 binds to both the pdx-1 B4/5 and insulin C1 elements.

Ins C1 can Substitute for B4/B5 to Drive Area II Activation in β Cells

Considering the similarity of B4/5 and Ins C1 binding in gel shift assays, it was surprising to find only modest sequence identity between the human (h) and mouse (m) B4/5 and mouse InsC1 (FIG. 15A). However, methylation interference assays over B4/5 suggested that the contact nucleotides of RIPE3b1 on InsC1and B4/5 were similar.(FIG. 15A; data not shown)). Because of sequence dissimilarity between B4/5 and Ins C1, it was determined whether Ins C1 could substitute for B4/5 in the context of the PstBstpTK reporter. Replacement of B4/5 with InsC1 maintained the same high level of activation found for wildtype PstBst in Min6 β cells, and like PstBst, there was no activity in NIH 3T3 cells (FIG. 17). Furthermore, mutants in B4/5 (B4mt, B5mt, B4+5 mt) and Ins C1 (mut3) that compromised complex A/RIPE3b1 binding also only reduced PstBst activity in Min6 cells. These data strongly suggested that the β cell-enriched RIPE3b1 transcription factor activates the B4/5 control element in Area II.

A Large Maf Transcription Factor Binds to Area II in Vivo

The RIPE3b1 transcription factor was recently isolated and shown to be a member of the large Maf (L-Maf) transcription factor family, most likely MafA (Matsuoka, et al., unpublished observations). To directly determine if RIPE3b1/Maf binds within Area II of the endogenous pdx-1 gene, a chromatin immunoprecipitation assay was performed using formaldehyde cross-linked chromatin from βTC-3 cells. Because Maf-A specific antiserum is unavailable, a polyclonal antiserum raised to N-terminal sequences conserved between members of the L-Maf family that recognizes Maf-A, Maf-B, and NRL, was used in this study. However, western blot analysis with specific c-Maf Maf-B, and NRL antisera suggests that MafA is the principal member of the family expressed in β cells (Matsuoka, et al., unpublished observations).

The DNA precipitated with the L-Maf antiserum was PCR amplified with Area II and phosphoenolpyruvate carboxykinase (PEPCK) promoter-specific primers. The L-Maf antibody was capable of immunoprecipitating Area II sequences, whereas the control IgG could not (FIG. 18). However, the Maf antiserum did not immunoprecipitate transcription control sequences from the PEPCK gene, which is not transcribed in β cells. These results demonstrate that RIPE3b1/Maf occupies the Area II region of the pdx-1 gene in β cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
U.S. Pat. No. 5,681,947
U.S. Pat. No. 5,652,099
U.S. Pat. No. 5,614,617
U.S. Pat. No. 5,670,663
U.S. Pat. No. 5,872,232
U.S. Pat. No. 5,859,221
U.S. Pat. No. 5,763,167
U.S. Pat. No. 5,446,137
U.S. Pat. No. 5,886,165
U.S. Pat. No. 5,714,606
U.S. Pat. No. 5,672,697
U.S. Pat. No. 5,466,786
U.S. Pat. No. 5,792,847
U.S. Pat. No. 5,223,618
U.S. Pat. No. 5,470,967
U.S. Pat. No. 5,378,825
U.S. Pat. No. 5,777,092
U.S. Pat. No. 5,623,070
U.S. Pat. No. 5,610,289
U.S. Pat. No. 5,602,240;
U.S. Pat. No. 5,858,988
U.S. Pat. No. 5,214,136
U.S. Pat. No. 5,700,922
U.S. Pat. No. 5,708,154
Basuyaux, J. P., et al., J. Biol. Chem. 272, 26188-26195 (1997)
Benkhelifa, S., et al., Oncogene 17, 247-254 (1998)
Blank, V., et al., Trends Biochem. Sci. 22, 437-441 (1997)
Callaerts, P., et al., Development 126, 4213-4222 (1999)
Carriere, C., et al., Cell Growth Differ. 6, 1531-1540 (1995)
Carriere, C., et al., Mol. Cell. Biol. 13,7257-7266 (1993)
Chau, K. Y., et al., J. Biol. Chem. 275, 37264-37270 (2000)
Chen, S., et al., Neuron 19, 1017-1030 (1997)
Cordes, S. P., et al., Cell 79, 1025-1034 (1994)
Cvekl, A., et al., BioEssays 18, 621-630 (1996)
Cvekl, A., et al., Mol. Cell. Biol. 14, 7363-7376 (1994)
Dahl, E., et al., BioEssays 19, 755-765 (1997)
Duncan, M. K., et al., J. Cell Sci. 113, 3173-3185 (2000)
Epstein, J., et al., J. Biol. Chem. 269, 8355-8361 (1994)
Epstein, J. A., et al., Genes Dev. 8, 2022-2034 (1994)
Furukawa, T., et al., Cell 91, 531-541 (1997)
German et al., Mol. Endocrinol., 5(2):292-9, 1991.
Haran, T. E., et al., J. Mol. Biol. 244, 135-143 (1994)
Hedge, S. P., et al., Mol. Cell. Biol. 18, 2729-2737 (1998)
Hemesath, T. J., et al., Genes Dev. 8, 2770-2780 (1994)
Herzig, S., et al., J. Biol. Chem. 275, 27989-27999 (2000)
Hussain, M. A., et al., J. Biol. Chem. 274, 28950-28957 (1999)
Hussain, M. A., et al., Mol. Cell. Biol. 17, 7186-7194 (1997)
Ishibashi, S., et al., Mech. Dev. 101, 155-166 (2001)
Jonat, C., et al., Cell 62, 1189-204 (1990)
Karlsson et al., Proc. Natl. Acad. Sci. USA, 84(24):8819-23, 1987.
Karlsson et al., Mol. Cell Biol., 9(2):823-7, 1989.
Kataoka, K., et al., J. Biol. Chem. 276, 819-826 (2001)
Kataoka, K., et al., J. Virol. 67, 2133-2141 (1993)
Kataoka, K., et al., Mol. Cell. Biol. 14, 700-712 (1994)
Kawauchi, S., et al., J. Biol. Chem. 274, 19254-19260 (2000)
Kerppola, T. K., et al., EMBO J 16, 2907-2916 (1997)
Kerppola, T. K., et al., Mol. Cell. Biol. 13, 5479-5489 (1993)
Kerppola, T. K., et al., Oncogene 9, 3149-3158 (1994)
Kerppola, T. K, et al., Oncogene 9, 675-684 (1994)
Klempnauer, K.-H., et al., Genes Dev. 3, 1582-1589 (1989)
Kurschner, C., et al., Biochem. Biophys. Res. Commun. 231, 333-339 (1997)
Laser, B., et al., J. Biol. Chem. 271, 28984-28994 (1996)
Larsson, L. I., et al., Mech. Dev. 79, 153-1599 (1998)
Liu, Q., et al., Oncogene 12, 207-211 (1996)
Martin, P., et al., Oncogene 7, 1721-1728 (1992)
Mitton, K. P., et al., J. Biol. Chem. 275, 29794-29799 (2000)
Naya et al., Genes Dev., 9(8):1009-19, 1995.
Nishizawa, M., et al., Proc. Natl. Acad. Sci. U.S.A. 86, 7711-7715 (1989)
Ogino, H., et al., Science 280, 115-118 (1998)
Ohlsson et al., EMBO. J., 12(11):4251-9, 1993.
Paolella, D. N., et al., Science 264, 1130-1133 (1994)
Peshavaria et al., Mol. Endocrinol., 8(6):806-16, 1994.
Petersen et al., FEBS Lett., 431(3):362-6, 1998.
Phillipe, J., et al., Mol. Cell. Biol. 11, 4877-4888 (1988)
Plaza, S., et al., Cell Growth Differ. 4, 1041-1050 (1993)
Planque, N., et al., J. Biol. Chem. 276, 29330-29337 (2001)
Plaza, S., et al., Cell Growth Differ. 8, 1115-1125 (1997)
Ring, B. Z., et al., Development 127, 307-317 (2000)
Ritz-Laser, B., et al., J. Biol. Chem. 274, 4124-4132 (1999)
Ritz-Laser, et al., J Biol. Chem. 275, 32708-32715 (2000)
St-Onge, L., et al., Nature 387, 406-409 (1997)
Sakai, M., et al., Nucleic Acids Res. 29, 1228-1237 (2001)
Sander, M., et al., Genes Dev. 11, 1662-1673 (1997)
Sharma et al., Mol. Endocrinol., 9(11):1468-76, 1995.
Sharma et al., Mol. Endocrinol., 9(9):1127-34, 1995.
Shieh et al., J. Biol. Chem., 266(25):16708-14, 1991.
Sieweke, M. H., et al., Cell 85, 49-60 (1996)
Turque, N., et al., Mol. Endocrinol. 8, 929-938 (1994)
Whelan et al., Mol. Cell Biol., 10(4):1564-72, 1990.
Wilson, D., et al., Genes Dev. 7, 2120-2134 (1993).

1. A method of generating a β-like cell comprising:
a) providing an expression cassette comprising a nucleic acid sequence encoding MafA under the control of a heterologous promoter; and
b) transferring the expression cassette into a non-insulin producing cell, wherein the expression of the MafA in the cell converts the cell into a β-like cell.

2. The method of claim 1, wherein the cell is further defined as a progenitor cell.

3. The method of claim 2, wherein the progenitor cell is further defined as a pancreatic progenitor cell.

4. The method of claim 1, wherein the cell is immortalized.

5. The method of claim 4, wherein the immortalized cell is susceptible to a cell kill agent.

6. The method of claim 5, wherein the immortalized cell further comprises a heterologous nucleic acid segment encoding a polypeptide that renders the cell susceptible to the cell kill agent.

7. The method of claim 6, wherein a nucleic acid segment encodes the cell kill agent and is under the control of an inducible promoter.

8. The method of claim 1, wherein the expression cassette is comprised in a non-viral vector.

9. The method of claim 8, wherein the non-viral vector is further defined as a plasmid.

10. The method of claim 1, wherein the expression cassette is comprised in a viral vector.

11. The method of claim 10, wherein the viral vector is an adenovirus, a retrovirus, a herpes-simplex virus, a vaccinia virus or an adeno-associated virus.

12. The method of claim 8, wherein the expression cassette is transferred into the cell by a non-viral delivery system.

13. The method of claim 12, wherein the non-viral delivery system is further defined as calcium phosphate precipitation, DEAE-dextran, electroporation, direct microinjection, DNA-loaded liposomes, cell sonification, gene bombardment using high velocity microprojectiles or receptor-mediated transfection.

14. The method of claim 1, wherein the promoter is further defined as an inducible promoter.

15. The method of claim 14, wherein the promoter promoter is a metallothonine promoter.

16. The method of claim 14, wherein the promoter is an inducible promoter.

17. The method of claim 16, further comprising providing to the cell an inducer of the promoter.

18. A method of generating a β-like cell comprising:
a) providing a MafA protein comprising a nuclear localization signal; and
b) contacting a cell with a sufficient amount of the MafA protein;
wherein contacting the cell with a sufficient amount of the MafA protein converts the cell into a β-like cell.

19. The method of claim 18, wherein the cell is further defined as a progenitor cell.

20. The method of claim 19, wherein the progenitor cell is further defined as a pancreatic progenitor cell.

21. The method of claim 18, wherein the cell is immortalized.

22. The method of claim 21, wherein the immortalized cell is susceptible to a cell kill agent.

23. The method of claim 22, wherein the immortalized cell further comprises a heterologous nucleic acid segment encoding a polypeptide that renders the cell susceptible to the cell kill agent.

24. The method of claim 22, wherein a nucleic acid segment encodes the cell kill agent and is under the control of an inducible promoter.

25. An implantable device for treating diabetes in a subject comprising:
a) a receptacle suitable for holding live cells; and
b) a cell-impermeable membrane operably fixed to the receptacle so as to confine the cells within the receptacle, wherein the membrane is permeable to insulin, regulatory signals that regulate the production of insulin and other factors necessary for the survival of the cells.

26. The device of claim 25, wherein the device is suitable for placement in a human body.

27. The device of claim 25, wherein the regulatory signal is glucose.

28. The device of claim 25, wherein the factors comprise nutrients.

29. A method of providing regulated insulin production to a subject comprising providing an effective amount of a composition comprising a β-like cell to a subject, wherein the β-like cell comprises an expression cassette comprising a nucleic acid sequence encoding MafA under the control of a heterologous promoter.

30. The method of claim 29, wherein the subject is a human.

31. The method of claim 29, wherein the heterologous promoter is an inducible promoter.

32. The method of claim 29, wherein the expression cassette is maintained in an episomal form or integrated into the cell genome.

33. A method of treating diabetes in a subject comprising providing an effective amount of a composition comprising a β-like cell to a subject, wherein the β-like cell comprises an expression cassette comprising a nucleic acid sequence encoding MafA under the control of a heterologous promoter.

34. The method of claim 33, wherein the subject is a human.

35. The method of claim 33, wherein the diabetes is Type I diabetes.

36. The method of claim 33, wherein the diabetes is Type II diabetes.

37. The method of claim 33, wherein the heterologous promoter is an inducible promoter.

38. The method of claim 33, wherein the expression cassette is maintained in an episomal form or integrated into the cell genome.

39. A composition comprising a β-like cell, wherein the β-like cell comprises an expression cassette comprising a nucleic acid sequence encoding MafA under the control of a heterologous promoter.

40. The method of claim 39, wherein the heterologous promoter is an inducible promoter.

41. The method of claim 39, wherein the expression cassette is maintained in an episomal form or integrated into the cell genome.


SEQUENCE LISTING (28 sequences)

SEQ ID NO: 1 - DNA, 1076 bp, Homo sapiens
  CDS (9)..(1067)
cccgggcgatggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccaccaccacgccgcccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtaggcgccggac

SEQ ID NO: 2 - protein, 352 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 3 - DNA, 1080 bp, Mus musculus
  CDS (1)..(1080)
atggccgcggagctggcgatgggcgcagagctgcccagcagcccactggccatcgagtacgtcaacgacttcgacctgatgaagttcgaggtgaagaaggagccgcccgaggccgagcgcttctgccaccgcctgccgcccggctcgctgtcctcgacgcccctcagcacgccctgctcctcggtgccctcttcgcccagcttctgcgcacccagcccgggcacaggcggcggcgcgggcggcgggggcagcgcggctcaggccgggggcgccccggggccgccgagtggaggccccggcactgtcgggggcgcctcaggaaaagcggtgctggaggatctgtactggatgagcgggtaccagcaccacctgaaccccgaggcgctcaacctgacgccggaggacgcggtggaggcgctcatcggcagcggccaccacggcgcgcaccacggcgcgcatcacccggcggctgctgcggcctatgaggccttccggggtcagagcttcgcgggcggcggcggcgcggacgacatgggtgccggccaccaccacggcgcacaccacactgcccaccatcatcactctgcccaccatcaccatcaccaccatcaccaccacggaggctctggccaccacggcggaggcgcgggtcacggcggaggcggcgcaggccaccacgtgcgcttggaggagcgcttctccgacgaccagctggtatccatgtccgtgcgggagctgaaccggcagctccgcggcttcagcaaggaggaggtcatccgactgaaacagaagcggcgcacgctcaagaaccgcggctacgcgcagtcgtgccgcttcaagcgggtgcagcagcggcacattctggagagcgagaagtgccagctccagagccaggtggagcagctgaagctggaggtggggcgtctggccaaggagcgggacctgtacaaggagaaatacgagaagttggcgggccggggcggccccgggggcgcgggcggggccggcttccctcgggagccctcgccagcgcaggctggccccggggcggccaaaggcgcacccgacttctttctgtga

SEQ ID NO: 4 - protein, 359 aa, Mus musculus
MAAELAMGAELPSSPLAIEYVNDFDLMKFEVKKEPPEAERFCHRLPPGSLSSTPLSTPCSSVPSSPSFCAPSPGTGGGAGGGGSAAQAGGAPGPPSGGPGTVGGASGKAVLEDLYWMSGYQHHLNPEALNLTPEDAVEALIGSGHHGAHHGAHHPAAAAAYEAFRGQSFAGGGGADDMGAGHHHGAHHTAHHHHSAHHHHHHHHHHGGSGHHGGGAGHGGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGGAGGAGFPREPSPAQAGPGAAKGAPDFFL

SEQ ID NO: 5 - DNA, 26 bp, Artificial Sequence
  Description of Artificial Sequence  Synthetic Primer
tggaaactgcagcttcagcccctctg

SEQ ID NO: 6 - DNA, 26 bp, Artificial Sequence
  Description of Artificial Sequence  Synthetic Primer
tggaaactgcagcttcagcccctctg

SEQ ID NO: 7 - DNA, 26 bp, Artificial Sequence
  Description of Artificial Sequence  Synthetic Primer
tggaaactgcagcttactaccctctg

SEQ ID NO: 8 - DNA, 26 bp, Artificial Sequence
  Description of Artificial Sequence  Synthetic Primer
tggaaactgcagcttcagcccctctg

SEQ ID NO: 9 - DNA, 24 bp, Artificial Sequence
  Description of Artificial Sequence  Synthetic Primer
tctttttgcaaagcacagcaaaaa

SEQ ID NO: 10 - DNA, 21 bp, Artificial Sequence
  Description of Artificial Sequence  Synthetic Primer
atggccgcggagctggcgatg

SEQ ID NO: 11 - DNA, 17 bp, Artificial Sequence
  Description of Artificial Sequence  Synthetic Primer
tcagaaagaagtcgggt

SEQ ID NO: 12 - DNA, 20 bp, Artificial Sequence
  Description of Artificial Sequence  Synthetic Primer
aggctttccggggtcagagt

SEQ ID NO: 13 - DNA, 20 bp, Artificial Sequence
  Description of Artificial Sequence  Synthetic Primer
tggagctggcacttctcgct

SEQ ID NO: 14 - DNA, 20 bp, Artificial Sequence
  Description of Artificial Sequence  Synthetic Primer
caacagctacccactagcca

SEQ ID NO: 15 - DNA, 20 bp, Artificial Sequence
  Description of Artificial Sequence  Synthetic Primer
ggcgagtttctcgcacttga

SEQ ID NO: 16 - DNA, 20 bp, Artificial Sequence
  Description of Artificial Sequence  Synthetic Primer
gtgcagcagagacacgtcct

SEQ ID NO: 17 - DNA, 18 bp, Artificial Sequence
  Description of Artificial Sequence  Synthetic Primer
caactagcaagcccactc

SEQ ID NO: 18 - DNA, 22 bp, Artificial Sequence
  Description of Artificial Sequence  Synthetic Primer
ggaactgtgaaacagtccaagg

SEQ ID NO: 19 - DNA, 21 bp, Artificial Sequence
  Description of Artificial Sequence  Synthetic Primer
ccccctggactttgctgtttg

SEQ ID NO: 20 - DNA, 22 bp, Artificial Sequence
  Description of Artificial Sequence  Synthetic Primer
gagtgacacctcacagctgtgg

SEQ ID NO: 21 - DNA, 22 bp, Artificial Sequence
  Description of Artificial Sequence  Synthetic Primer
ggcaggcctttggatcatagcc

SEQ ID NO: 22 - DNA, 26 bp, Artificial Sequence
  Description of Artificial Sequence Synthetic Primer
agctttctttttgcaaagcacagcat

SEQ ID NO: 23 - DNA, 26 bp, Artificial Sequence
  Description of Artificial Sequence Synthetic Primer
agcttaaagcacagcaaaaatattat

SEQ ID NO: 24 - DNA, 29 bp, Artificial Sequence
  Description of Artificial Sequence Synthetic Primer
agcttctttttgcaaagcacagcaaaaat

SEQ ID NO: 25 - DNA, 22 bp, Artificial Sequence
  Description of Artificial Sequence Synthetic Primer
ggtgggaaatccttccctcaag

SEQ ID NO: 26 - DNA, 21 bp, Artificial Sequence
  Description of Artificial Sequence Synthetic Primer
ccttagggatagaccccctgc

SEQ ID NO: 27 - DNA, 22 bp, Artificial Sequence
  Description of Artificial Sequence Synthetic Primer
gagtgacacctcacagctgtgg

SEQ ID NO: 28 - DNA, 22 bp, Artificial Sequence
  Description of Artificial Sequence Synthetic Primer
ggcaggcctttggatcatagcc